SEQUENCE LISTING (34 sequences)

SEQ ID NO: 1 - DNA, 1668 bp, Artificial sequence
  Synthetic
atgaagcgctcgaagcgcttcgcggtgctggcccagcgcccggtgaaccaagatggcctcatcggggagtggcccgaagagggcctcatcgcaatggactcgccgttcgatcccgtgtcctcggtgaaggtcgataacggcctgatcgtggagctggacggcaagcgccgcgaccagttcgatatgatcgaccggttcattgcggactacgcgatcaatgtggaacgcaccgaacaggcgatgcgcctggaagcggtcgagatcgcccggatgctcgtggacatccatgtgagccgcgaagagatcatcgcgatcaccacggcgatcaccccggccaaagccgtggaagtgatggcccagatgaacgtcgtcgagatgatgatggcgctgcagaagatgcgcgcccgccgcaccccgtccaaccagtgccatgtcaccaacctgaaggataacccggtgcagatcgccgcggacgcggccgaggccggcatccggggcttctcggaacaggaaaccaccgtgggcattgcccgctacgcccccttcaacgcgctggccctgctggtcggctcgcagtgcggccggccgggcgtgctgacccagtgcagcgtggaagaagcgaccgagctggagctgggcatgcgcggcctgacctcgtacgcggaaaccgtgtcggtctacgggaccgaggccgtctttaccgacggcgacgacacgccgtggtccaaggcctttctggcgagcgcctatgccagccgcggcctgaagatgcggtacacgagcggcaccggctccgaggccctgatgggctacagcgagtcgaagtccatgctgtatctggagtcccggtgcatcttcatcacgaagggcgcgggcgtgcaagggctgcagaatggcgccgtgtcgtgcatcggcatgaccggcgcggtgcccagcggcatccgcgcggtgctcgccgaaaacctgattgcctccatgctggacctggaagtcgcgagcgcgaacgaccagacgttcagccacagcgacatccgccgcacggcgcgcacgctgatgcagatgctgccgggcaccgacttcatcttcagcggctactccgcggtgccgaactatgataatatgttcgccggcagcaacttcgatgccgaggatttcgacgactacaacatcctgcagcgcgatctgatggtcgatggcgggctgcgccccgtcaccgaagcggaaaccatcgccatccgccagaaagccgcgcgggccatccaggccgtgttccgcgagctggggctgccgccgatcgccgacgaagaagtcgaggccgccacctacgcgcacggctccaatgaaatgcccccgcgcaacgtcgtggaggacctgtcggcggtggaagagatgatgaagcgcaacatcaccggcctggacatcgtcggcgcgctgtcgcgcagcggcttcgaggacatcgcgagcaatatcctgaacatgctgcgccaacgcgtgaccggcgactacctccagacctcggcgattctggaccgccagtttgaggtcgtgtcggccgtgaacgacatcaacgactaccagggcccgggcacgggctaccgcatctcggccgagcgctgggccgagatcaagaacatcccgggcgtggtgcagccggacacgatcgagtga

SEQ ID NO: 2 - protein, 555 aa, Klebsiella pneumonia
MKRSKRFAVLAQRPVNQDGLIGEWPEEGLIAMDSPFDPVSSVKVDNGLIVELDGKRRDQFDMIDRFIADYAINVERTEQAMRLEAVEIARMLVDIHVSREEIIAITTAITPAKAVEVMAQMNVVEMMMALQKMRARRTPSNQCHVTNLKDNPVQIAADAAEAGIRGFSEQETTVGIARYAPFNALALLVGSQCGRPGVLTQCSVEEATELELGMRGLTSYAETVSVYGTEAVFTDGDDTPWSKAFLASAYASRGLKMRYTSGTGSEALMGYSESKSMLYLESRCIFITKGAGVQGLQNGAVSCIGMTGAVPSGIRAVLAENLIASMLDLEVASANDQTFSHSDIRRTARTLMQMLPGTDFIFSGYSAVPNYDNMFAGSNFDAEDFDDYNILQRDLMVDGGLRPVTEAETIAIRQKAARAIQAVFRELGLPPIADEEVEAATYAHGSNEMPPRNVVEDLSAVEEMMKRNITGLDIVGALSRSGFEDIASNILNMLRQRVTGDYLQTSAILDRQFEVVSAVNDINDYQGPGTGYRISAERWAEIKNIPGVVQPDTIE

SEQ ID NO: 3 - DNA, 1668 bp, Artificial sequence; identical to SEQ ID NO: 1
  Synthetic

SEQ ID NO: 4 - protein, 426 aa, Klebsiella pneumoniae [HC1]
ATGAGCGAGAAAACCATGCGCGTGCAGGATTATCCGTTAGCCACCCGCTGCCCGGAGCATATCCTGACGCCTACCGGCAAACCATTGACCGATATTACCCTCGAGAAGGTGCTCTCTGGCGAGGTGGGCCCGCAGGATGTGCGGATCTCCCGCCAGACCCTTGAGTACCAGGCGCAGATTGCCGAGCAGATGCAGCGCCATGCGGTGGCGCGCAATTTCCGCCGCGCGGCGGAGCTTATCGCCATTCCTGACGAGCGCATTCTGGCTATCTATAACGCGCTGCGCCCGTTCCGCTCCTCGCAGGCGGAGCTGCTGGCGATCGCCGACGAGCTGGAGCACACCTGGCATGCGACAGTGAATGCCGCCTTTGTCCGGGAGTCGGCGGAAGTGTATCAGCAGCGGCATAAGCTGCGTAAAGGAAGCTAA

SEQ ID NO: 5 - protein, 141 aa, Klebsiella pneumonia
MSEKTMRVQDYPLATRCPEHILTPTGKPLTDITLEKVLSGEVGPQDVRISRQTLEYQAQIAEQMQRHAVARNFRRAAELIAIPDERILAIYNALRPFRSSQAELLAIADELEHTWHATVNAAFVRESAEVYQQRHKLRKGS

SEQ ID NO: 6 - DNA, 1824 bp, Artificial sequence
  Synthetic
atgccgttaatagccgggattgatatcggcaacgccaccaccgaggtggcgctggcgtccgacgacccgcaggcgagggcgtttgttgccagcgggatcgttgcgacgacgggcatgaaagggacgcgggacaatatcgccgggaccctcgccgcgctggagcaggccctggcgaaaacaccgtggtcgatgagcgatgtctctcgcatctatcttaacgaagccgcgccggtgattggcgatgtggcgatggagaccatcaccgagaccattatcaccgaatcgaccatgatcggtcataacccgcagacgccgggcggggtgggcgttggcgtggggacgactatcgccctcgggcggctggcgacgctgccggcggcgcagtatgccgaggggtggatcgtactgattgacgatgccgtcgatttccttgacgccgtgtggtggctcaatgaggcgctcgaccgggggatcaacgtggtggcggcgatccttaaaaaggacgacggcgtgctggtgaacaaccgcctgcgtaaaaccctgccggtggtggatgaagtgacgctgctggagcaggtccccgagggggtgatggcggcggtggaagtggccgcgccgggccaggtggtgcggatcctgtcgaatccctacgggatcgccaccttcttcgggctaagcccggaagagacccaggccatcgtccccatcgcccgcgccctgattggcaaccgttccgcggtggtgctcaagaccccgcagggggacgtgcagtcgcgggtgatcccggcgggcaacctctacattagcggcgaaaagcgccgcggagaggccgatgttgccgagggcgcggaagccatcatgcaggcgatgagcgcctgcgctccggtacgcgacatccgcggcgaaccgggcactcacgccggcggcatgcttgagcgggtgcgcaaggtaatggcgtccctgaccgaccatgagatgagcgcgatatacatccaggatctgctggcggtggatacgtttattccgcgcaaggtgcagggcgggatggccggcgagtgcgccatggaaaatgccgtcgggatggcggcgatggtgaaagcggatcgtctgcaaatgcaggttatcgcccgcgaactgagcgcccgactgcagaccgaggtggtggtgggcggcgtggaggccaacatggccatcgccggggcgttaaccactcccggctgtgcggcgccgctggcgatcctcgacctcggcgccggctcgacggatgcggcgatcgtcaacgcggaggggcagataacggcggtccatctcgccggggcggggaatatggtcagcctgttgattaaaaccgagctgggcctcgaggatctttcgctggcggaagcgataaaaaaatacccgctggccaaagtggaaagcctgttcagtattcgtcatgagaatggcgcggtggagttctttcgggaagccctcagcccggcggtgttcgccaaagtggtgtacatcaaggagggcgaactggtgccgatcgataacgccagcccgctggaaaaaattcgtctcgtgcgccggcaggcgaaagagaaagtgtttgtcaccaactgcctgcgcgcgctgcgccaggtctcacccggcggttccattcgcgatatcgcctttgtggtgctggtgggcggctcatcgctggactttgagatcccgcagcttatcacggaagccttgtcgcactatggcgtagtcgccgggcagggcaatattcggggaacagaagggccgcgcaatgcggtcgccaccgggctgctactggccggtcaggcgaattaa

SEQ ID NO: 7 - protein, 607 aa, Klebsiella pneumonia
MPLIAGIDIGNATTEVALASDYPQARAFVASGIVATTGMKGTRDNIAGTLAALEQALAKTPWSMSDVSRIYLNEAAPVIGDVAMETITETIITESTMIGHNPQTPGGVGVGVGTTIALGRLATLPAAQYAEGWIVLIDDAVDFLDAVWWLNEALDRGINVVAAILKKDDGVLVNNRLRKTLPVVDEVTLLEQVPEGVMAAVEVAAPGQVVRILSNPYGIATFFGLSPEETQAIVPIARALIGNRSAVVLKTPQGDVQSRVIPAGNLYISGEKRRGEADVAEGAEAIMQAMSACAPVRDIRGEPGTHAGGMLERVRKVMASLTGHEMSAIYIQDLLAVDTFIPRKVQGGMAGECAMENAVGMAAMVKADRLQMQVIARELSARLQTEVVVGGVEANMAIAGALTTPGCAAPLAILDLGAGSTDAAIVNAEGQITAVHLAGAGNMVSLLIKTELGLEDLSLAEAIKKYPLAKVESLFSIRHENGAVEFFREALSPAVFAKVVYIKEGELVPIDNASPLEKIRLVRRQAKEKVFVTNCLRALRQVSPGGSIRDIAFVVLVGGSSLDFEIPQLITEALSHYGVVAGQGNIRGTEGPRNAVATGLLLAGQAN

SEQ ID NO: 8 - DNA, 2558 bp, Artificial sequence
  Synthetic
acttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctagaaataattttgtttaactttaaaaggaggtatatcgatgcccctgatcgccggcattgatatcggcaacgcgaccacggaggtcgcgctggcgtccgattatccccaggcccgggccttcgtggcgtccggcatcgtcgccaccaccggcatgaagggcacgcgggacaacatcgccggcacactcgccgccctggagcaggcgctggccaagaccccgtggagcatgtcggacgtgagccgcatctacctgaacgaagcggccccggtgatcggcgatgtggcgatggaaaccattaccgaaacgattattaccgagtccaccatgatcggccataacccgcagacgccggggggggtgggcgtgggcgtgggcaccacgattgcgctggggcgcctggccaccctccccgcggcgcagtatgccgaagggtggattgtgctgatcgatgatgcggtggatttcctcgacgcggtctggtggctgaatgaggcgctggatcgcgggatcaatgtcgtggcggcgatcctcaagaaagatgacggcgtgctcgtgaataaccgcctgcgcaagacgctccccgtggtggacgaagtgaccctgctggaacaggtgccggagggcgtcatggccgcggtcgaagtggcggcccccggccaggtcgtgcgcatcctcagcaacccgtacggcatcgccacgttcttcggcctcagcccggaggaaacccaggcgatcgtcccgatcgcccgcgcgctgatcgggaaccgctcggcggttgtcctgaaaaccccgcagggggatgtgcagagccgcgtgatccccgccggcaacctgtatatcagcggcgaaaagcgccgcggcgaagccgacgtggccgagggcgccgaagccatcatgcaagccatgagcgcgtgcgccccggtccgcgatatccggggcgagcccggcacccacgcgggcggcatgctggaacgcgtccggaaggtgatggcctcgctgacggaccacgagatgtcggcgatctatatccaggatctgctcgccgtggacacgtttatcccgcggaaagtccagggcggcatggccggcgagtgcgcgatggagaacgccgtgggcatggcggcgatggtgaaggccgatcgcctgcagatgcaagtcatcgcccgggaactgagcgcgcgcctgcagaccgaagtggtcgtcgggggggtcgaggcgaacatggcgattgcgggcgcgctgacgacgcccgggtgcgcggcgccgctggccattctcgacctgggcgcgggctccaccgacgcggcgattgtgaatgcggagggccagatcaccgcggtccacctggcgggcgcgggcaacatggtcagcctcctgatcaagaccgaactgggcctggaagatttgagcctggccgaagccatcaagaagtacccgctggcgaaggtcgaaagcctgtttagcatccgccatgagaatggcgccgtggagttctttcgcgaggcgctctcccccgccgtgttcgccaaagtcgtgtacatcaaggaaggggagctggtgccgatcgacaatgcgtcgccgctggaaaagatccgcctggtccgccgccaggccaaggagaaggtgttcgtgacgaactgcctgcgcgcgctgcgccaagtgtcgccgggcggctcgatccgcgacatcgccttcgtggtcctggtggggggctcctcgctggatttcgaaatcccgcaactgatcaccgaagcgctctcgcactacggggtcgtcgcgggccagggcaacatccgcggcaccgagggcccccgcaacgcggtcgccaccggcctgctgctggccggccaggccaactgaaaaggaggtatatcgatgtcgctgagcccgccgggcgtccgcctgttctatgacccccgcggccatcacgccggggccatcaatgaactgtgctggggcctggaagaacagggcgtgccctgccagaccatcacgtacgacggcggcggcgacgcggcggcgctgggcgccctcgccgcccggagctccccgctgcgcgtgggcatcggcctgagcgcctcgggcgagatcgccctgacgcacgcgcagctgaccgcggatgccccgctcgccaccgggcacgtgacggattcggacgaccatctgcgcaccctgggcgcgaacgcgggccaactggtgaaggtcctcccgctgtccgagcgcaactga

SEQ ID NO: 9 - protein, 607 aa, Klebsiella pneumonia
MPLIAGIDIGNATTEVALASDYPQARAFVASGIVATTGMKGTRDNIAGTLAALEQALAKTPWSMSDVSRIYLNEAAPVIGDVAMETITETIITESTMIGHNPQTPGGVGVGVGTTIALGRLATLPAAQYAEGWIVLIDDAVDFLDAVWWLNEALDRGINVVAAILKKDDGVLVNNRLRKTLPVVDEVTLLEQVPEGVMAAVEVAAPGQVVRILSNPYGIATFFGLSPEETQAIVPIARALIGNRSAVVLKTPQGDVQSRVIPAGNLYISGEKRRGEADVAEGAEAIMQAMSACAPVRDIRGEPGTHAGGMLERVRKVMASLTDHEMSAIYIQDLLAVDTFIPRKVQGGMAGECAMENAVGMAAMVKADRLQMQVIARELSARLQTEVVVGGVEANMAIAGALTTPGCAAPLAILDLGAGSTDAAIVNAEGQITAVHLAGAGNMVSLLIKTELGLEDLSLAEAIKKYPLAKVESLFSIRHENGAVEFFREALSPAVFAKVVYIKEGELVPIDNASPLEKIRLVRRQAKEKVFVTNCLRALRQVSPGGSIRDIAFVVLVGGSSLDFEIPQLITEALSHYGVVAGQGNIRGTEGPRNAVATGLLLAGQAN

SEQ ID NO: 10 - protein, 117 aa, Klebsiella pneumonia
MSLSPPGVRLFYDPRGHHAGAINELCWGLEEQGVPCQTITYDGGGDAAALGALAARSSPLRVGIGLSASGEIALTHAQLTADAPLATGHVTDSDDHLRTLGANAGQLVKVLPLSERN

SEQ ID NO: 11 - DNA, 1488 bp, Artificial sequence
  Synthetic
atgaattttcatcatctggcttactggcaggataaagcgttaagtctcgccattgaaaaccgcttatttattaacggtgaatatactgctgcggcggaaaatgaaacctttgaaaccgttgatccggtcacccaggcaccgctggcgaaaattgcccgcggcaagagcgtcgatatcgaccgtgcgatgagcgcagcacgcggcgtatttgaacgcggcgactggtcactctcttctccggctaaacgtaaagcggtactgaataaactcgccgatttaatggaagcccacgccgaagagctggcactgctggaaactctcgacaccggcaaaccgattcgtcacagtctgcgtgatgatattcccggcgcggcgcgcgccattcgctggtacgccgaagcgatcgacaaagtgtatggcgaagtggcgaccaccagtagccatgagctggcgatgatcgtgcgtgaaccggtcggcgtgattgccgccatcgtgccgtggaacttcccgctgttgctgacttgctggaaactcggcccggcgctggcggcgggaaacagcgtgattctaaaaccgtctgaaaaatcaccgctcagtgcgattcgtctcgcggggctggcgaaagaagcaggcttgccggatggtgtgttgaacgtggtgacgggttttggtcatgaagccgggcaggcgctgtcgcgtcataacgatatcgacgccattgcctttaccggttcaacccgtaccgggaaacagctgctgaaagatgcgggcgacagcaacatgaaacgcgtctggctggaagcgggcggcaaaagcgccaacatcgttttcgctgactgcccggatttgcaacaggcggcaagcgccaccgcagcaggcattttctacaaccagggacaggtgtgcatcgccggaacgcgcctgttgctggaagagagcatcgccgatgaattcttagccctgttaaaacagcaggcgcaaaactggcagccgggccatccacttgatcccgcaaccaccatgggcaccttaatcgactgcgcccacgccgactcggtccatagctttattcgggaaggcgaaagcaaagggcaactgttgttggatggccgtaacgccgggctggctgccgccatcggcccgaccatctttgtggatgtggacccgaatgcgtccttaagtcgcgaagagattttcggtccggtgctggtggtcacgcgtttcacatcagaagaacaggcgctacagcttgccaacgacagccagtacggccttggcgcggcggtatggacgcgcgacctctcccgcgcgcaccgcatgagccgacgcctgaaagccggttccgtcttcgtcaataactacaacgacggcgatatgaccgtgccgtttggcggctataagcagagcggcaacggtcgcgacaaatccctgcatgcccttgaaaaattcactgaactgaaaaccatctggataagcctggaggcctga

SEQ ID NO: 12 - protein, 495 aa, E. coli
MNFHHLAYWQDKALSLAIENRLFINGEYTAAAENETFETVDPVTQAPLAKIARGKSVDIDRAMSAARGVFERGDWSLSSPAKRKAVLNKLADLMEAHAEELALLETLDTGKPIRHSLRDDIPGAARAIRWYAEAIDKVYGEVATTSSHELAMIVREPVGVIAAIVPWNFPLLLTCWKLGPALAAGNSVILKPSEKSPLSAIRLAGLAKEAGLPDGVLNVVTGFGHEAGQALSRHNDIDAIAFTGSTRTGKQLLKDAGDSNMKRVWLEAGGKSANIVFADCPDLQQAASATAAGIFYNQGQVCIAGTRLLLEERIADEFLALLKQQAQNWQPGHPLDPATTMGTLIDCAHADSVHSFIREGESKGQLLLDGRNAGLAAAIGPTIFVDVDPNASLSREEIFGPVLVVTRFTSEEQALQLANDSQYGLGAAVWTRDLSRAHRMSRRLKAGSVFVNNYNDGDMTVPFGGYKQSGNGRDKSLHALEKFTELKTIWISLEA

SEQ ID NO: 13 - DNA, 1491 bp, Artificial sequence
  Synthetic
atgatgaattttcagcacctggcttactggcaggaaaaagcgaaaaacctggccattgaaacgcgcttatttattaacggcgaatattgcgccgcggccgataataccacctttgagactatcgaccccgccgcgcagcagacattagcccaggtcgcccgcggtaaaaaagccgacgtcgaacgggcggtgaaagccgcgcgccaggcttttgataacggcgactggtcgcaggcctcccccgcacagcgtaaagcgatcctcactcgctttgctaatctgatggaggcccatcgtgaagagctggcgctgctggaaacgctggataccggcaagccgattcgccacagcctgcgcgacgatattcccggcgccgcccgcgccattcgctggtatgccgaagcgctggataaagtctatggcgaagtggcccccaccggcagcaacgagctggcgatgatcgttcgcgaaccaattggcgtgatcgccgcggtggtgccgtggaacttcccgctgctgctggcctgctggaaactcggcccggcgctggcggcaggcaatagcgtaatcctcaaaccctcggaaaaatcgccgcttaccgccctgcgtctggccgggctggcgaaagaggccggcctgccggacggcgtgttgaacgtggtcagcggctttggccacgaggccgggcaggcgctggccctgcatcctgatgttgaagtcatcaccttcaccggctccacccgcaccggcaagcagctgctgaaagacgccggcgacagcaatatgaagcgcgtgtggctggaagcgggcggcaagagcgccaacattgtcttcgccgattgcccggatctgcaacaagcggttcgcgccaccgccggcggcatcttctacaaccagggacaggtgtgcatcgccgggacccgtctgctgctcgaggagagcatcgctgacgagttcctggcgcggctgaaagctgaggcgcaacactggcagccgggcaacccgctcgatccggacaccaccatgggcatgctgattgacaatacccatgccgacaacgtgcatagctttattcgcggcggcgaaagccaaagcaccctgttcctcgacggacggaaaaacccgtggcctgccgccgttggcccgaccattttcgttgacgtcgacccggcatcaaccctcagccgggaagagatcttcggcccggtgctggtggtgacccgcttcaaaagcgaagaagaggcgctaaagctcgccaatgacagcgactacggcttgggcgccgcggtgtggacccgcgatctctcccgcgcccaccgcatgagccgccgcctgaaggccggctcggtcttcgtcaacaactataacgatggtgatatgaccgttccgttcggcggctacaagcagagcggcaacgggcgcgataaatcgctgcacgcgctggaaaaattcaccgaactgaaaaccatctggattgccctggagtcttga

SEQ ID NO: 14 - protein, 496 aa, Klebsiella pneumonia
MMNFQHLAYWQEKAKNLAIETRLFINGEYCAAADNTTFETIDPAAQQTLAQVARGKKADVERAVKAARQAFDNGDWSQASPAQRKAILTRFANLMEAHREELALLETLDTGKPIRHSLRDDIPGAARAIRWYAEALDKVYGEVAPTGSNELAMIVREPIGVIAAVVPWNFPLLLACWKLGPALAAGNSVILKPSEKSPLTALRLAGLAKEAGLPDGVLNVVSGFGHEAGQALALHPDVEVITFTGSTRTGKQLLKDAGDSNMKRVWLEAGGKSANIVFADCPDLQQAVRATAGGIFYNQGQVCIAGTRLLLEESIADEFLARLKAEAQHWQPGNPLDPDTTMGMLIDNTHADNVHSFIRGGESQSTLFLDGRKNPWPAAVGPTIFVDVDPASTLSREEIFGPVLVVTRFKSEEEALKLANDSDYGLGAAVWTRDLSRAHRMSRRLKAGSVFVNNYNDGDMTVPFGGYKQSGNGRDKSLHALEKFTELKTIWIALES

SEQ ID NO: 15 - DNA, 1176 bp, Artificial sequence
  Synthetic
atgtctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattag

SEQ ID NO: 16 - protein, 391 aa, S. cerevisiae
MSAAADRLNLTSGHLNAGRKRSSSSVSLKAAEKPFKVTVIGSGNWGTTIAKVVAENCKGYPEVFAPIVQMWVFEEEINGEKLTEIINTRHQNVKYLPGITLPDNLVANPDLIDSVKDVDIIVFNIPHQFLPRICSQLKGHVDSHVRAISCLKGFEVGAKGVQLLSSYITEELGIQCGALSGANIATEVAQEHWSETTVAYHIPKDFRGEGKDVDHKVLKALFHRPYFHVSVIEDVAGISICGALKNVVALGCGFVEGLGWGNNASAAIQRVGLGEIIRFGQMFFPESREETYYQESAGVADLITTCAGGRNVKVARLMATSGKDAWECEKELLNGQSAQGLITCKEVHEWLETCGSVEDFPLFEAVYQIVYNNYPMKNLPDMIEELDLHED

SEQ ID NO: 17 - DNA, 753 bp, Artificial sequence
  Synthetic
atgggattgactactaaacctctatctttgaaagttaacgccgctttgttcgacgtcgacggtaccattatcatctctcaaccagccattgctgcattctggagggatttcggtaaggacaaaccttatttcgatgctgaacacgttatccaagtctcgcatggttggagaacgtttgatgccattgctaagttcgctccagactttgccaatgaagagtatgttaacaaattagaagctgaaattccggtcaagtacggtgaaaaatccattgaagtcccaggtgcagttaagctgtgcaacgctttgaacgctctaccaaaagagaaatgggctgtggcaacttccggtacccgtgatatggcacaaaaatggttcgagcatctgggaatcaggagaccaaagtacttcattaccgctaatgatgtcaaacagggtaagcctcatccagaaccatatctgaagggcaggaatggcttaggatatccgatcaatgagcaagacccttccaaatctaaggtagtagtatttgaagacgctccagcaggtattgccgccggaaaagccgccggttgtaagatcattggtattgccactactttcgacttggacttcctaaaggaaaaaggctgtgacatcattgtcaaaaaccacgaatccatcagagttggcggctacaatgccgaaacagacgaagttgaattcatttttgacgactacttatatgctaaggacgatctgttgaaatggtaa

SEQ ID NO: 18 - protein, 250 aa, S. cerevisiae
MGLTTKPLSLKVNAALFDVDGTIIISQPAIAAFWRDFGKDKPYFDAEHVIQVSHGWRTFDAIAKFAPDFANEEYVNKLEAEIPVKYGEKSIEVPGAVKLCNALNALPKEKWAVATSGTRDMAQKWFEHLGIRRPKYFITANDVKQGKPHPEPYLKGRNGLGYPINEQDPSKSKVVVFEDAPAGIAAGKAAGCKIIGIATTFDLDFLKEKGCDIIVKNHESIRVGGYNAETDEVEFIFDDYLYAKDDLLKW

SEQ ID NO: 19 - protein, 542 aa, C. necator
MKVITAREAAALVQDGWTVASAGFVGAGHAEAVTEALEQRFLQSGLPRDLTLVYSAGQGDRGARGVNHFGNAGMTASIVGGHWRSATRLATLAMAEQCEGYNLPQGVLTHLYRAIAGGKPGVMTKIGLHTFVDPRTAQDARYHGGAVNERARQAIAEGKACWVDAVDFRGDEYLFYPSFPIHCALIRCTAADARGNLSTHREAFHHELLAMAQAAHNSGGIVIAQVESLVDHHEILQAIHVPGILVDYVVVCDNPANHQMTFAESYNPAYVTPWQGEAAVAEAEAAPVAAGPLDARTIVQRRAVMELARRAPRVVNLGVGMPAAVGMLAHQAGLDGFTLTVEAGPIGGTPADGLSFGASAYPEAVVDQPAQFDFYEGGGIDLAILGLAELDGHGNVNVSKFGEGEGASIAGVGGFINITQSARAVVFMGTLTAGGLEVRAGDGGLQIVREGRVKKIVPEVSHLSFNGPYVASLGIPVLYITERAVFEMRAGADGEARLTLVEIAPGVDLQRDVLDQCSTPIAVAQDLREMDARLFQAGPLHL

SEQ ID NO: 20 - DNA, 1628 bp, Artificial sequence
  Synthetic
atgaaggtgatcaccgcacgcgaagcggcggcactggtgcaggacggctggaccgtggccagcgcgggcttgtcggcgccggccatgccgaggccgtgaccgaggcgctggagcagcgcttcctgcagagcgggctgccgcgcgacctgacgctggtgtactcggccgggcagggcgaccgcggcgcgcgcggcgtgaaccacttcggcaatgccggcatgaccgccagcatcgtcggcggccactggcgctcggccacgcggctggccacgctggccatggccgagcagtgcgagggctacaacctgccgcagggcgtgctgacgcacctataccgcgccatcgccggcggcaagcccggcgtgatgaccaagatcggcctgcacaccttcgtcgacccgcgcaccgcgcaggatgcgcgctaccacggcggcgccgtcaacgagcgcgcgcgccaggccattgccgagggcaaggcatgctgggtcgatgcggtcgacttccgcggcgacgaatacctgttctacccgagcttcccgatccactgcgcgctgatccgctgcaccgcggccgacgcccgcggcaacctcagcacccatcgcgaagccttccaccatgagctgctggcgatggcgcaggcggcccacaactcgggcggcatcgtgatcgcgcaggtggaaagcctggtcgaccaccacgagatcctgcaggccatccacgtgcccggcatcctggtcgactacgtggtggtctgcgacaaccccgccaaccaccagatgacgtttgccgagtcctacaacccggcctacgtgacgccatggcaaggcgaggcagcggtggccgaagcggaagcggcgccggtggctgccggcccgctcgacgcgcgcaccatcgtgcagcgccgtgcggtgatggaactggcgcgccgtgcgccgcgcgtggtcaacctgggcgtgggcatgccggcagcggtcggcatgctggcgcaccaggccgggctggacggcttcacgctgaccgtcgaggccggccccatcggcggcacgcccgcggatggcctcagcttcggtgcctcggcctacccggaggcggtggtggatcagcccgcgcagttcgatttctacgagggcggcggcatcgacctggccatcctcggcctggccgagctggatggccacggcaacgtcaatgtcagcaagttcggcgaaggcgagggcgcatcgattgccggcgtcggcggctttatcaacatcacgcagagcgcgcgcgcggtggtgttcatgggcacgctgacggcgggcgggctggaagtccgcgccggcgacggcggcctgcagatcgtgcgcgaaggccgcgtgaagaagatcgtgcctgaggtgtcgcacctgagcttcaacgggccctatgtggcgtcgctcggcatcccggtgctgtacatcaccgagcgcgcggtgttcgagatgcgcgctggcgcagacggcgaagcccgcctcacgctggtcgagatcgcccccggcgtggacctgcagcgcgacgtgctcgaccagtgctcgacgcccatcgccgttgcgcaggacctgcgcgaaatggatgcgcggctgttccaggccgggcccctgcacctgtaa

SEQ ID NO: 21 - protein, 630 aa, C. necator
MTASHAVHARSLADPEGFWAEQAARIDWETPFGQVLDNSRAPFTRWFVGGRTNLCHNAVDRHLAARASQPALHWVSTETDQARTFTYAELHDEVSRMAAILQGLDVQKGDRVLIYMPMIPEAAFAMLACARIGAIHSVVFGGFASVSLAARIEDARPRVVVSADAGSRAGKVVPYKPLLDEAIRLSSHQPGKVLLVDRQLAQMPRTEGRDEDYAAWRERVAGVQVPCVWLESSEPSYVLYTSGTTGKPKGVQRDTGGYAVALATSMEYIFCGKPGDTMFTASDIGWVVGHSYIVYGPLLAGMATLMYEGTPIRPDGGILWRLVEQYKVNLMFSAPTAIRVLKKQDPAWLTRYDLSSLRLLFLAGEPLDEPTARWIQDGLGKPVVDNYWQTESGWPILAIQRGIEALPPKLGSPGVPAYGYDLKIVDENTGAECPPGQKGVVAIDGPLPPGCMSTVWGDDDRFVRTYWQAVPNRLCYSTFDWGVRDADGYVFILGRTDDVINVAGHRLGTREIEESLSSNAAVAEVAVVGVQDALKGQVAMAFCIARDPARTATAEARLALEGELMKTVEQQLGAVARPARVFFVNALPKTRSGKLLRRAMQAVAEGRDPGDLTTIEDPGALEQLQAALKG

SEQ ID NO: 22 - DNA, 1893 bp, Artificial sequence
  Synthetic
atgacggcaagccatgccgtgcatgcccgttcgctggccgaccccgaggggttctgggccgaacaggcggcgcgcatcgactgggaaaccccgttcggccaggtgctcgacaacagccgcgcgccctttacgcgctggttcgtcggcgggcgcaccaacctgtgccacaacgcggtcgaccgccacctggcggcccgcgccagccagccggcgctgcactgggtctcgaccgagaccgaccaggcccgcacctttacctacgccgagctgcacgacgaagtcagccgcatggccgcgatcctgcagggcctggacgtgcagaagggcgaccgcgtgctgatctacatgccgatgatcccggaagccgcctttgccatgctggcctgcgcgcgcatcggcgcgatccattcggtggtgttcggcggctttgcctcggtcagcctggccgcgcgcatcgaggatgcccggccgcgcgtggtggtcagcgccgacgccggctcgcgtgccggcaaggtggtgccctacaagccgctgctggacgaggccatccggctctcgtcgcaccagcccgggaaggtgctgctggtggaccggcaactggcgcaaatgccccgtaccgagggccgcgatgaggactacgccgcctggcgcgaacgcgtggccggcgtgcaggtgccgtgcgtgtggctggaatcgagcgagccgtcgtacgtgctatacacctccggcaccaccggcaagcccaagggcgtgcagcgcgataccggcggctacgcggtggcgctggccacctcgatggaatacatcttctgcggcaagcccggcgacaccatgttcaccgcgtcggacatcggctgggtggtggggcacagctatatcgtctacggcccgctgctggccggcatggccacgctgatgtatgaaggcacgccgatccgccccgacggtggcatcctgtggcggctggtggagcaatacaaggtcaacctgatgttcagcgcgccgaccgcgatccgcgtgctgaagaagcaggacccggcctggctgacccgctacgacctgtccagcctgcgcctgctgttcctggccggcgagccgctggacgagcccaccgcgcgctggatccaggacggcctgggcaagcccgtggtcgacaactactggcagaccgaatccggctggccgatcctcgcgatccagcgcggcatcgaggcgctgccgcccaagctgggctcgcccggcgtgcccgcctacggctatgacctgaagatcgtcgacgagaacaccggcgctgaatgcccgccggggcagaagggtgtggtcgccatcgacggcccgctgccgccgggatgcatgagcacggtctggggcgacgacgaccgcttcgtgcgcacctactggcaggcggtgccgaaccggctgtgctattcgaccttcgactggggcgtgcgcgacgccgacggctatgtttttatcctgggccgcaccgacgacgtgatcaacgttgccggccaccggctgggcacccgcgagatcgaggaaagcctgtcgtccaacgctgccgtggccgaggtggcggtggtgggcgtgcaggacgcgctcaaggggcaggtggcgatggccttctgcatcgcccgcgatccggcgcgcacggccacggccgaagcgcggctggcattggagggcgagttgatgaagacggtggagcagcaactgggtgccgtggcgcggccggcgcgcgtattctttgtcaatgcactgcccaagacccgctccggcaagttgctgcggcgcgccatgcaggcggtggccgaagggcgcgatccgggcgacctgaccacgatcgaggacccgggtgcgctggaacagttgcaggcagcgctgaaaggctag

SEQ ID NO: 23 - protein, 576 aa, C. necator
MAAAALPASRRDDYRALYESFRWEIPPHFNIAEACCGRWARDPATMDRIAVYTEHEDGRRNAHTFAHIQAEANRLSAALRALGVARGDRVAIVMPQRIETVIAHMAIYQLGAIAMPLSMLFGPEALAYRIAHSEANVAIADETSIDNVLAARPECPTLATVIAAGGAHGRGDHDWDVLLAAQLPTFVAEQTKADEAAVLIYTSGTTGPPKGALIPHRALIGNLTGFVCSQNWYPQDDDVFWSPADWAWTGGLWDALMPALYFGKPIVGYQGRFSAERAFELLERYAVTNTFLFPTALKQMMKACPEPRQRYDIRLRALMSAGEAVGETVFGWCRDALGVIVNEMFGQTEINYIVGNCTAQNDDKQLGWPARPGSMGRPYPGHRVQVIDDEGQPCAPGEDGEVAVCATDSAGHPDPVFFLGYWKNEAATAGKYAERDGLRWCRTGDLARVDADGYLWYQGRADDVFKSSGYRIGPSEIENCLLKHPAVSNCAVVPSPDPERGAVVKAFVVLTPSVARSFDGDAALVTELQAHVRGQLAPYEYPKAIEFIDQLPMTTTGKIQRRVLRLLEEARAGKRA

SEQ ID NO: 24 - DNA, 1731 bp, Artificial sequence
  Synthetic
atggccgcagctgcgttgccggcaagccggcgcgacgactatcgcgccctgtatgaatccttccgctgggaaatccccccgcatttcaatatcgccgaggcctgctgcgggcgctgggcgcgcgacccggccacgatggaccgcatcgcggtctataccgagcatgaggacggccgccgcaacgcgcatacctttgcccatatccaggccgaagccaaccgcctgtcggcggcgctgcgcgcactgggcgtggcgcgcggcgaccgcgtggcaatcgtgatgccgcagcggatcgagaccgtgatcgcgcatatggcgatctaccagctcggcgccatcgccatgccgctgtcgatgctgttcgggcccgaggcgctggcctaccgtatcgcacacagcgaagccaatgtggcgatcgcggacgagacttccatcgacaatgtgctggccgcgcgcccggaatgcccgacgctggccaccgtgattgccgccggcggcgcgcatggccgcggcgaccacgactgggacgtgctgctggccgcgcagctgccgacttttgtcgccgagcagaccaaggccgacgaggccgcggtgctgatctacaccagcggcaccaccggcccgcccaagggcgcgctgatcccgcaccgcgcgctgatcggcaacctgaccggctttgtctgctcgcagaactggtatccgcaggacgacgacgtgttctggagcccggccgactgggcctggaccggcggcctgtgggatgcgctgatgccggcgctgtatttcggcaagcccatcgtcggctaccagggccgcttctccgccgagcgcgccttcgagctgctggagcgctacgccgtcaccaacaccttcctgttcccgaccgcgctcaagcagatgatgaaggcctgccccgagccgcggcagcgctacgacatcaggctgcgtgcgctgatgagcgccggcgaggccgtgggcgagaccgtgttcggctggtgccgcgatgcgctgggcgtgatcgtcaacgagatgttcggccagaccgagatcaactacatcgtcggcaactgcaccgcgcagaacgacgacaagcagctgggctggccggcacgaccgggctcgatggggcgtccctatccgggccaccgcgtgcaggtgatcgacgacgaaggccagccctgcgcgccgggcgaggacggcgaggtcgcggtatgcgccaccgacagcgccgggcatccggacccggtgttcttcctcggctactggaagaacgaagccgccaccgcgggcaagtacgccgagcgcgacggcctgcgctggtgccgcaccggcgacctggcgcgcgtcgatgccgatggctacctgtggtaccaggggcgtgccgacgatgtgttcaagtcctcgggctaccgcatcgggccgagcgagatcgagaactgcctgctcaagcatccggcggtgtccaactgcgccgtggtgccctcgcccgaccccgagcgcggcgccgtggtcaaggccttcgtggtgctgacaccgtcggtggcgcgctcgttcgacggcgacgcggcgctggtcacggagctgcaggcgcatgtgcgcggccagctggcgccgtatgaatacccgaaggcgatcgaattcatcgaccagctgccgatgaccaccaccggcaagatccagcggcgcgtgctgcgcttgctggaggaagcgcgcgcgggcaagcgcgcctag

SEQ ID NO: 25 - protein, 685 aa, C. necator
MSEGKAPRHAAQQELADVSEAEIAVHWPEEDYVPPAGQFIAQANLTDPHIFERFSLERFPECFKEFADLLDWYKYWETTLDTSNPPFWRWFVGGRINACHNCVDRHLAAYRNKTAIHFVPEPEDEAVHHLTYQELFVRVNELAALLREFCGLKAGDRVTLHMPMVAELPITMLACARIGVIHSQVFSGFSGKACAERIADSESRLLITMDAYHRGGELLDHKEKADIAVAEAASAGQQVEKVLIWQRYPGKYSSAALLVKGRDVILNDVLAGFRGRRVEPEPMPAEAPLFLMYTSGTTGRPKGCQHSTGGYLSYVAWTSKYIQDIHPEDVYWCMADIGWITGHSYIVYGPLALAASSVVYEGVPTWPDAGRPWRIAESLGVNIFHTSPTAIRALRRNGPDEPAKYDCHFKHMTTVGEPIEPEVWKWYHREVGKGEAVIVDTWWQTENGGFLCSTLPGIHPMKPGSTGPGIPGIHPVIFDEEGNEVPAGSGKAGNICIRNPWPGIFQTVWKDPDRYVRQYYARYCKNPDSKDWHDWPYMAGDGAMQAADGYFRILGRIDDVINVSGHRLGTKEIESAALLVPDVAEAAVVPVADEVKGKVPDLYVSLKPGLSPSIKIANKVSAAVVSQIGAIARPHRVVIVPDMPKTRSGKIMRRVLAAISNHQEPGDVSTLANPEVVEKIRELAT

SEQ ID NO: 26 - DNA, 2058 bp, Artificial sequence
  Synthetic
atgtctgaaggcaaagcgccacgccatgctgcccagcaggaattggccgatgtgtccgaggccgaaatcgcggtccattggcccgaggaggactatgtcccgccggccggccagttcattgcgcaggccaatctgaccgatccccatattttcgagcgcttctccctcgaacgtttccccgagtgcttcaaggagttcgcagacctgctggactggtacaaatactgggaaacgaccctggataccagcaacccgcctttctggcgctggttcgtcggcggcaggatcaacgcctgccacaattgcgtggatcgccacctcgctgcatacaggaacaagaccgcgattcatttcgtgcccgagccggaggatgaggcggtgcatcacctcacctaccaggagctcttcgttcgcgtcaatgagctggccgccctgctgcgcgagttctgcggcctgaaggccggcgaccgcgtcacgctgcatatgccgatggtggccgaactgcccatcaccatgctcgcctgcgcccgcatcggcgtgattcattcgcaggtattcagcggcttcagcggcaaggcctgcgccgagcgcatcgcggactccgagagccggctgctgatcaccatggacgcctatcaccgcggcggtgaattgctcgatcacaaggaaaaggccgacatcgccgtggcagaagccgccagcgccggtcagcaggtcgagaaggtcctgatctggcagcgctacccgggcaagtattccagtgccgccctactggtgaagggccgcgatgtcattctcaatgacgtgctcgccgggttccgcggcaggcgtgtcgagcccgagccgatgccggcggaggcgccgctgttcctgatgtacacgagcggcaccacgggccggcccaagggctgccagcattccactggcggctatctgtcctatgtggcgtggacctctaagtacatccaggatatccaccccgaggacgtctactggtgcatggccgatattggctggatcaccgggcattcctacatcgtctatggcccgctcgcgctcgccgcttcgtctgtcgtctatgaaggcgtgccgacctggcccgacgccggccggccctggcgtattgcggaaagccttggcgtcaatatcttccacacctcgcccaccgcaatccgcgcgctgcggcgcaacgggcccgacgagccggcgaagtacgactgccatttcaagcacatgaccacggtgggcgagccgatcgagcccgaagtctggaagtggtaccaccgtgaagtcggcaaaggcgaggcggtgatcgtggacacctggtggcaaaccgagaatggcggcttcctctgcagcacgctgccgggcatccacccgatgaagcccggcagcactggcccgggaatcccgggcattcatccggtgatctttgacgaggaaggcaatgaggtcccggccggctcgggcaaggcgggcaacatctgcatccgcaatccctggccgggcatattccagaccgtctggaaggatccggaccgctacgtgcgccagtactatgcgcgctattgcaagaatcccgacagcaaggactggcacgactggccgtatatggcgggcgatggcgcaatgcaggcggcggacggctactttcgcatccttggccgcatcgacgacgtgatcaatgtttccggccatcgcctcggcaccaaggagatcgaatccgcagcactgctggtgccggacgtcgccgaggcggcggtggtgccggtggccgacgaggtcaagggcaaggtgcctgatctctatgtatcgctcaagccgggactgtcgccctccatcaagatcgcgaacaaggtctcggccgcggtggtatcccagattggcgcgattgcgcgtccgcatcgggtcgtgatcgtccccgacatgcccaagacacgctcgggcaagatcatgcgccgcgtgctggcggcgatctccaaccaccaggagcctggcgacgtatccacgcttgccaatccggaggtcgtcgagaagatcagggagctggcgacatag

SEQ ID NO: 27 - protein, 660 aa, C. necator [sequence omitted]

SEQ ID NO: 28 - DNA, 1983 bp, Artificial sequence
  Synthetic
atgtccgccatcgaatcggtgatgcaagagcatcgcgtgttcaacccgcccgaaggcttcgccagccaggccgcgatccccagcatggaggcctaccaggcgctgtgcgacgaagccgagcgtgactatgaaggtttctgggcgcgccacgcgcgcgagctgctgcactggaccaagcccttcaccaaggtgctggaccaaagcaacgcaccgttctacaagtggttcgaagacggcgagctcaacgcctcttacaactgcctggaccgcaatctgcagaacggcaatgcggacaaggtcgcgatcgtgttcgaggccgacgacggcagcgtgacgcgcgtcacctaccgcgagctgcatggcaaggtgtgccgcttcgccaacggcctgaaggcgctcggcatcaggaagggcgaccgcgtggtgatctacatgccgatgtcggtcgaaggcgtggtcgcgatgcaggcctgcgcacgcctgggcgccacgcactcggtggtgttcggcggcttctcggccaagtcgctgcaggagcggctggtggacgtgggcgcggtggcgctgatcaccgccgacgagcagatgcgcggcggcaaggcgctgccgctcaaggccatcgccgatgacgcgctggcgctgggcggctgcgaggccgtcaggaacgtgatcgtctaccgccgcaccggcggcaaggttgcctggaccgaaggccgcgaccgctggatggaagatgtcagcgccggccagccggatacctgcgaagccgagccggtgagcgccgagcacccgctgttcgtgctctacacctccggctccaccggcaagcccaagggcgtgcagcacagcaccggcggctacctgctgtgggcgctgatgacaatgaagtggaccttcgacatcaagcccgacgacctgttctggtgtaccgcggacatcggctgggtcaccggccacacctatattgcctacggcccgctggccgcgggcgccacccaggtggtgttcgaaggcgtgccgacctaccccaacgccggccgcttctgggacatgatcgcgcgccacaaggtcagcatcttctacaccgcgccgaccgcgatccgctcgctgatcaaggccgccgaggccgacgagaagatccacccgaaacagtacgacctgtccagcctgcgcctgctcggcaccgtgggcgagccgatcaaccccgaagcctggatgtggtactacaagaacatcggcaacgagcgctgcccgatcgtcgacaccttctggcagaccgagaccggcggccacatgatcacgccgctgccgggcgcgacgccgctggtgccgggttcgtgcacgctgccgctgccgggcatcatggccgccatcgtcgacgagaccggccatgacgtgcccaacggcaacggcggcatcctggtggtcaagcgtccgtggccggccatgatccgcaccatctggggcgatccggagcgcttcaggaagagctacttccccgaagagctcggcggcaagctctacctggccggcgacggctcgatccgcgacaaggacaccggctacttcaccatcatgggccgcatcgacgacgtgctgaacgtgtcgggccaccgcatggggacgatggagatcgagtccgcgctggtgtccaacccgctggtggctgaagccgccgtggtgggccgccccgacgacatgaccggcgaggccatctgcgccttcgtcgtgctcaagcgttcgcgtccgactggcgaagaggccgtcaagatcgcgacggagctgcgcaactgggtcggcaaggagatcggcccgatcgccaagcccaaggacatccgctttggcgacaacctgcccaagacgcgctcgggcaagatcatgcggcgcctgctgcggtcgctggccaagggggaggagatcacgcaggacacctcgacgctggagaatccggccatcctggagcagctcaagcaggcgcagtga

SEQ ID NO: 29 - protein, 714 aa, C. necator
MSTRDLYTHAQLRRLFHPRTIAVVGATPNARSFAGRAMTNLQQFDGNVLLVNPRYPEVNGQVCYPSLSALPEAPDCVLIATARETVEPIVRECAGLGVGGVVLFASGYAETGNPEQIAEQARLVAIARESGMLLLGPNSIGYANYINHALVSFTPLPARGGELPAHAIGLVSQSGALAFALEQAANHGTAFSHVFSCGNACDIDVTDQIAYLAGDPSCAAIACVFEGLSDASRIIRAAQVCAEAGKPLVVYKMARGTAGAAAAMSHTGSMAGSDRAYSTALREAGVVQVDTIEQLVPTTVFFAKAPRPTTSGVAIVSGSGGAGIVAADEAERFNVPLPQPCDATRAVLESHIPDFGAARNPCDLTAQAANNFDSFIQCGDAVFADPAYGAAVVPLVVTGDGNGRRFQVFNDLAVKHGKMACGLWMSNWMEGPEAVESEALPRLALFRSVSHCFAALAAWQAREQWLLSRATPKPPRLTHASVAAEARARIVAAPADTLTEREAKDVLAMYGVPVVGESLATSEQDAVRAADACGYPVVLKVESPAIPHKSEAGVIRLGVNSAQEVAVAYREVMANARKVTADDRINGVLVQSQVPTGIEILVGARVDPHLGALLVVGLGGVMVELMQDTVATIAPCSAQQARAMLEQLRGVALLKGFRGAAGVDMDLLAEIVASLSEFAADQRDVIAEFDVNPLICTPDRIVAVDALIERRVGA

SEQ ID NO: 30 - DNA, 2145 bp, Artificial sequence
  Synthetic
atgtcgacacgcgatctctatacccacgcgcaactgcggcgcctcttccatccgcgcaccatcgcggtggtcggcgcgacgccgaacgctcgctcgttcgccggccgggccatgacgaacctgcagcagttcgacggcaacgtgctgctggtcaacccccgctaccccgaggtgaacgggcaggtctgctatccgtcgctgtcggcgctgcccgaggcgcccgactgcgtgctgatcgccaccgcgcgcgaaacggtggagcccatcgtgcgcgagtgcgcggggctgggcgtgggcggcgtggtgctgttcgcgtcgggctatgccgagaccggcaatccggagcagattgccgagcaggctcggctggtcgccattgcccgggaaagcggcatgctgctgctcggtccgaacagcatcggctatgcgaactacatcaaccatgcgctggtgtcgttcacgccgctgcccgcgcgtggcggcgaactgccggcccatgcgatcgggctggtcagccagtccggcgcgctggcatttgcgctggaacaggcggccaaccacggcacggcgttcagccacgtgttctcgtgcggcaatgcgtgcgatatcgacgtgaccgaccagatcgcctatctcgccggggatccctcgtgcgcggcgatcgcatgcgtattcgaagggctgtccgacgccagccggatcattcgcgcggcgcaagtctgcgcggaagccggcaagccgctggtggtctacaagatggcgcgcgggacggcgggcgcggcggcggccatgtcgcataccggctcgatggcgggatccgaccgcgcctacagcacggcgctgcgcgaagctggcgtggtgcaggtcgataccatcgagcagctcgtgccgacgacggtgttcttcgccaaggccccccggccgacgacgtccggcgtggccatcgtctcgggttcgggcggcgcgggcattgtcgccgccgacgaggccgagcgtttcaacgtgccgctgccgcagccgtgtgacgcgacccgcgccgtgctcgaatcgcacattcctgacttcggcgccgcgcgcaacccgtgcgacctgaccgcccaggccgccaacaacttcgactccttcatccagtgcggcgacgcggtcttcgccgatcccgcctacggcgccgccgtggtgccgctggtggtgaccggcgacggcaacggccgccgcttccaggtgttcaacgacctagccgtcaagcacggcaagatggcgtgcggcctgtggatgtcgaactggatggaagggccggaggcggtcgagtccgaggcgctgccgcgccttgcgctgttccgctcggtctcgcactgcttcgcggcgctggccgcgtggcaggcacgggagcaatggctgttgtcgcgcgccacgccgaagccgccgcgcctgacacacgcttcggtggccgccgaagcgcgcgcgcgcatcgttgccgcgccggccgatacgctcaccgagcgtgaagccaaggacgtccttgccatgtacggcgtgccggtggtgggcgagtccctggcgacgagcgagcaggacgccgtgcgcgccgccgatgcctgcggctatccggtcgtgctgaaggtcgagagcccggccatcccgcacaagtcggaagcgggcgtgatccgcctcggcgtgaactcggcgcaggaggttgccgtcgcgtaccgcgaggtcatggcgaatgcgcgcaaggtgaccgccgacgaccgcatcaacggcgtgctggtgcagagccaggtgccgaccggcatcgagatcttggtcggcgcccgcgtggacccgcacctcggcgcgctgctggtggtggggctgggcggggtgatggtcgagctgatgcaggacacggtcgcgaccatcgcgccgtgctcggcgcagcaggcgcgcgccatgctggagcagctgcgcggcgtggcgctgctgaagggcttccgcggcgcggcgggcgtggacatggacctgctggcggaaatcgtcgccagcctgtccgagttcgcggcggaccagcgcgacgtgatcgccgagttcgatgtgaatccgctgatctgcacgccggaccgcatcgtggcggtggatgcgctgatcgaacggagagtgggggcctga

SEQ ID NO: 31 - protein, 660 aa, C. necator
MTSIQSVVHEGRMFPPSRHASAKAAIPSMEAYQALCDEAERDYEGFWARHARELLHWTKPFTKVLDQSNAPFYKWFEDGELNASYNCLDRNLQNGNADKVAIVFEADDGSVTRVTYRELHGKVCRFANGLKALGIRKGDRVVIYMPMSVEGVVAMQACARLGATHSVVFGGFSAKSLQERLVDVGAVALITADEQMRGGKALPLKPIADDALALGGCEAVRNVIVYRRTGGKVAWTEGRDRWMEDVSAGQPETCEAEPVSAEHPLFVLYTSGSTGKPKGVQHSTGGYLLWALMTMKWTFDIKPDDLFWCTADIGWVTGHTYIAYGPLAAGATQVVFEGVPTYPNAGRFWDMIARHKVSIFYTAPTAIRSLIKAAEADEKIHPKQYDLSSLRLLGTVGEPINPEAWMWYYKNIGNERCPIVDTFWQTETGGHMITPLPGATPLVPGSCTLPLPGIMAAIVDETGHDVPNGNGGILVVKRPWPAMIRTIWGDPERFRKSYFPEELGGKLYLAGDGSIRDKDTGYFTIMGRIDDVLNVSGHRMGTMEIESALVSNPLVAEAAVVGRPDDMTGEAICAFVVLKRSRPTGEEAVKIATELRNWVGKEIGPIAKPKDIRFGDNLPKTRSGKIMRRLLRSLAKGEEITQDTSTLENPAILEQLGQAR

SEQ ID NO: 32 - DNA, 1983 bp, Artificial sequence
  Synthetic
atgacaagcattcaatccgttgtgcacgaagggcggatgttcccgccatcccgccacgccagcgctaaggccgcgattcccagcatggaggcctaccaggcactgtgcgacgaagccgagcgtgactatgaaggtttctgggcgcgccacgcgcgcgagctgctgcactggaccaagcccttcaccaaggtgctggaccaaagcaacgcaccgttctacaagtggttcgaagacggcgagctcaacgcctcttacaactgcctggaccgcaatctgcagaacggcaatgcggacaaggtcgcgatcgtgttcgaggccgacgacggcagcgtgacgcgcgtcacctaccgcgagctgcatggcaaggtgtgccgctttgccaacggcctgaaggcgctcggcatcaggaagggcgaccgcgtggtgatctacatgccgatgtcggtcgaaggcgtggtcgcgatgcaggcctgcgcacgcctgggcgccacgcactcggtggtgttcggcggcttctcggccaagtcgctgcaggagcggctggtggacgtgggcgcggtggcgctgatcaccgccgacgagcagatgcgcggcggcaaggcgctgccgctcaagcccatcgccgatgacgcgctggcgctggggggctgcgaggccgtcaggaacgtgatcgtctaccgccgcaccggcggcaaggttgcctggaccgaaggccgcgaccgctggatggaagatgtcagcgccggccagccggagacctgcgaagccgagccggtgagcgccgagcacccgctgttcgtgctctacacctccggctccaccggcaagcccaagggcgtgcagcacagcaccggcggctacctgctgtgggcgctgatgacaatgaagtggaccttcgacatcaagcccgacgacctgttctggtgtaccgcggacatcggctgggtcaccggccacacctatattgcctacggcccgctggccgcgggcgccacccaggtggtgttcgaaggcgtgccgacctaccccaacgccggccgcttctgggacatgatcgcgcgccacaaggtcagcatcttctacaccgcgccgaccgcgatccgctcgctgatcaaggccgccgaggccgacgagaagatccacccgaaacagtacgacctgtccagcctgcgcctgctcggcaccgtgggcgagccgatcaaccccgaagcctggatgtggtactacaagaacatcggcaacgagcgctgcccgatcgtcgacaccttctggcagaccgagaccggcggccacatgatcacgccgctgccgggcgcgacgccgctggtgccgggttcgtgcacgctgccgctgccgggcatcatggccgccatcgtcgacgagaccggccatgacgtgcccaacggcaacggcggcatcctggtggtcaagcgtccgtggccggccatgatccgcaccatctggggcgatccggagcgcttcaggaagagctacttccccgaagagctcggcggcaagctctacctggccggcgacggctcgatccgcgacaaggacaccggctacttcaccatcatgggccgcatcgacgacgtgctgaacgtgtcgggccaccgcatggggacgatggagatcgagtccgcgctggtgtccaacccgctggtggccgaagccgccgtggtgggccgccccgacgacatgaccggcgaggccatctgcgccttcgtcgtgctcaagcgttcgcgtccgactggcgaagaggccgtcaagatcgcgacggagctgcgcaactgggtcggcaaggagatcggcccgatcgccaagcccaaggacatccgctttggcgacaacctgcccaagacgcgctcgggcaagatcatgcggcgcctgctgcggtcgctggccaagggggaggagatcacgcaggacacctcgacgctggagaatccggccatcctggagcagcttggccaggcacgctga

SEQ ID NO: 33 - protein, 550 aa, C. necator
MRDYAQAFDGFSYDDAVARQLHGSQEAMNACVECCDRHALPGRIALFWEGRDGNSRSWTFTELQALSAQFAGFLKAQGVQPGDRVAGLLPRNAELLVTILGTWRAGAVYQPLFTAFGPKAIEHRLNASGAKVVVTDGANRPKLDDVDGCPAIVTVAGDKGRGLVRGDFSFWAELERQPASFEPVPRRGDDPFLMMFTSGTTGPAKPLLVPLKAIAAFAGYMSDAVDLRAEDAFWNLADPGWAYGLYYAVTGPLALGHPTTFYDGPFTVESTCRVIRKYGITNLAGSPTAYRLLIAAGEAVSGPLRGRLRAVSSAGEPLNPEVIRWFASELGVTIHDHYGQTELGMVLCNHHALAHPVRMGAAGFASPGHRVVVVDDEQRELPPGRPGTLALDLKRSPMCWFGGYHGTPTSGFAGGYYLTGDSAELNDDGSISFIGRADDVITTSGYRVGPFDVESALIEHPAVVEAAVIGKPDPERTELIKAFVVLDPQYRAAPELAEALRQHVRKRLAAHAYPREIEFVVELPKTPSGKVQRFILRNQEVARAREAAAA

SEQ ID NO: 34 - DNA, 1653 bp, Artificial sequence
  Synthetic
atgcgcgactacgcccaagccttcgacggattttcctatgacgacgccgtggcacggcaactgcacggcagccaggaggcaatgaacgcctgcgtcgaatgctgcgaccgccacgcgctgccgggccgtatcgcgctgttctgggaagggcgagacggcaattcgcgcagctggacctttaccgagctgcaggcactgtccgcgcagtttgccggcttcctgaaggcgcagggcgtgcagccgggcgaccgcgtggcgggcctgctgccgcgcaatgcggaactgctggtgacgattctcggcacctggcgcgccggcgcggtgtaccagccgctgttcacggccttcggccccaaggccatcgagcaccggctcaatgcgtccggcgcgaaggttgtggtcaccgatggcgccaaccgccccaagctggatgacgtggatggctgtcccgccattgtcaccgtggccggcgacaagggccgcggcctggtgcgcggcgacttcagcttctgggccgaactggaacgccagccggcgtcgttcgagccggtgccgcgccggggcgacgaccccttcctgatgatgttcacctccggcaccaccggcccggccaagccgctgctggtgccgctcaaggccattgccgcgtttgccggctatatgagcgacgcggtcgacctgcgcgcggaagacgctttctggaacctggccgatccgggctgggcctatggcctgtattacgcggtcacgggcccgctggcgctgggccatcccaccaccttctacgatggcccgttcaccgtggagagcacatgccgtgtgatccgcaagtacggcatcaccaacctggccggctcgcccacggcataccggctgctgatcgccgcgggcgaggccgtgtcaggcccgctgcgcgggcggctgcgcgcggtcagcagcgcgggcgagccgctcaacccggaagtgatccgctggttcgccagcgagctgggcgtgaccatccacgaccactacggccagaccgagctgggcatggtgctgtgcaaccaccatgcgctggcgcatccggtgcgcatgggcgcggccggctttgccagccccgggcaccgcgtggtggtggtggacgatgaacagcgcgaactgccgccgggccggccgggcacgctggcgctggacctgaagcgctcgccgatgtgctggttcggcggctatcacggcacgcccaccagcgggtttgccggcggctactacctgaccggcgattccgccgagctgaatgacgacggcagcatcagcttcataggccgggccgacgacgtcatcaccacctctggctaccgcgtgggcccgttcgacgtggaaagcgcgctgatcgagcacccggccgtggtcgaggccgcggtgatcggcaagcccgatccggagcgcaccgagctgatcaaggcctttgtcgtgctggacccgcaatatcgcgccgcgccggaactggccgaggcgctgcgccagcacgtgcgtaagcgcctggccgcccatgcctacccgcgcgagatcgagttcgtcgtcgagctgcccaagacccccagcggcaaggtccagcgctttatcctgcgcaaccaggaagtggcccgcgcgcgcgaggcggccgctgcctga

(19) United States
(12) Patent Application Publication
(10) Pub. No.: US 2019/0233853 A1
(43) Pub. Date: Aug. 1, 2019

(54) METHODS AND MATERIALS FOR THE BIOSYNTHESIS OF BETA HYDROXY ACIDS AND/OR DERIVATIVES THEREOF AND/OR COMPOUNDS RELATED THERETO

(71) Applicant: INVISTA NORTH AMERICA S.A.R.L., Wilmington, DE (US)

(72) Inventors: Cristina Serrano Amatriain, Redcar (GB); Alexander Brett Foster, Redcar (GB); Stephen Thomas Cartman, Redcar (GB)

(21) Appl. No.: 16/264,765

(22) Filed: Feb. 1, 2019

Related U.S. Application Data
(60) Provisional application No. 62/659,306, filed on Apr. 18, 2018.
(60) Provisional application No. 62/625,066, filed on Feb. 1, 2018.
(60) Provisional application No. 62/625,013, filed on Feb. 1, 2018.

(51) Int. Cl. C12P 7/52; C12N 9/88; C12N 9/02; C12N 9/16

(52) U.S. Cl. CPC C12P 7/52; C12N 9/88; C12Y 402/0103; C12Y 301/03021; C12Y 102/01003; C12N 9/16; C12N 9/0008

(57) ABSTRACT

Methods and materials for the production of beta hydroxy acids, such as 3-hydroxypropanoic acid (3-HP) and/or derivatives thereof and/or compounds related thereto, are provided. Also provided are products produced in accordance with these methods and materials.

This patent application claims the benefit of priority from U.S. Provisional Application Ser. No. 62/659,306 filed Apr. 18, 2018, U.S. Provisional Application Ser. No. 62/625,066 filed Feb. 1, 2018 and U.S. Provisional Application Ser. No. 62/625,013 filed Feb. 1, 2018, the contents of each of which are incorporate herein by reference in their entireties.

### FIELD

The present invention relates to biosynthetic methods and materials for the production of beta hydroxy acids, such as 3-hydroxypropanoic acid (3-HP), and/or derivatives thereof and/or other compounds related thereto. The present invention also relates to products biosynthesized or otherwise encompassed by these methods and materials.

Replacement of traditional chemical production processes relying on, for example fossil fuels and/or potentially toxic chemicals, with environmentally friendly (e.g., green chemicals) and/or “cleantech” solutions is being considered, including work to identify building blocks suitable for use in the manufacturing of such chemicals. See, “Conservative evolution and industrial metabolism in Green Chemistry”, Green Chem., 2018, 20, 2171-2191.

3-HP has been identified as a value-added platform compounds among renewable biomass production products proposed by the United States Department of Energy (Werpy, T. & Petersen, G. US DOE, Washington, D C, 2004). For example, 3-HP has versatile applications in but not limited to, conversion to bulk chemicals such as acrylic acid (see WO 2013/192451), 1,3-propanediol, 3-hydroxypropionaldehyde and malonic acid as well as plastics (Valdehuesa et al. Appl. Microbiol. Biotechnol. 2013 97:3309-3321) and in the polymerization and formation of biodegradable materials.

Several microbes that are able to naturally produce 3-HP have been identified (Kumar et al. Biotechnol Adv. 2013 31:945-961). However, low yield of 3-HP has reportedly restricted commercialization.

3-HP synthesis from glycerol comprises two reactions catalyzed by a glycerol dehydratase leading to 3-hydroxypropionaldehyde (3-HPA), and an aldehyde dehydrogenase converting 3-HPA into 3-HP. In the facultative anaerobe Klebsiella pneumoniae, under reductive conditions, glycerol is metabolized to 1,3-propanediol with 3-HPA as the intermediate. In this organism, dhaB1, dhaB2 and dhaB3 encode the three subunits of the enzyme that catalyzes the first reaction (see biocyc with the extension .org/META/NEW-IMAGE?type=ENZYME& object=CPLX-3581 of the world wide web). This enzyme is vitamin B₁₂-dependent and is inactivated by glycerol during catalysis with the cofactor being irreversibly damaged (Ashok et al. Appl. Microbiol. Biotechnol. 2011 990:1253-1265). The enzyme can also be inactivated by oxygen in the absence of substrate (Ashok et al. Appl. Microbiol. Biotechnol. 2011 990:1253-1265). However, this organism has a reactivator of this enzyme, a diol dehydratase reactivase encoded by gdrA and gdrB (Kajiura et al. The Journal of Biological Chemistry 2001 276: 36514-36519). This enzyme exchanges the modified coenzyme, cyanocobalamin (CN-Cbl), by adenosylcobalamin (AdoCbl) in an ATP- and Mg²⁺-dependent reaction.

A NAD+-dependent gamma-glutamyl-gamma-aminobutyraldehyde dehydrogenase, encoded by puuC classified in EC 1.2.1.3, which can catalyze the conversion of 3-HPA into 3-HP when overexpressed, has also been described in K. pneumoniae (Ashok et al. Appl. Microbiol. Biotechnol. 2011 990:1253-1265).

In E. coli, the same reaction can be catalyzed by the product of gene aldH (NAD+-dependent aldehyde dehydrogenase) (Jo et al. Appl Microbiol Biotechnol 2008 81: 51).

Various approaches have been described for 3-HP production from glycerol in Klebsiella pneumoniae (Ashok et al. Appl. Microbiol. Biotechnol. 2011 990:1253-1265; Huang et al. Bioresource Technology 2013 128: 505-512; Ko et al. Bioresource Technology 2017 244(Part 1):1096-1103) and E. coli (Raj et al. Process Biochemistry 2008 43(12): 1440-1446; Raj et al. Appl Microbiol Biotechnol 2009 84:649) by the overexpression of dhaB from K. pneumoniae, and either puuC from K. pneumoniae or aldH from E. coli. Such methods have reportedly reached levels of 40 g/L in fed-batch processes. However, while K. pneumoniae can synthesize vitamin B₁₂ under anaerobic or microaerobic conditions, supplementation of media with this expensive vitamin is necessary in the recombinant strains of E. coli which can be inconvenient in large volume fermentations. Also, growth of these strains is done in microaerobic conditions.

Expression of the glycerol dehydratase reactivase, encoded by gdrAB, permits the performance of the assay in aerobic conditions (Jiang et al. Biotechnol. Biofuels 2016 9:57).

3-HP production from glucose and xylose has been developed as well using Corynebacterium glutamicum as platform strain. In this organism, glycerol is produced from dihydroxyacetone phosphate by dephosphorylation followed by reduction. However, levels of glycerol produced are very low and heterologous expression of glycerol 3-phosphate dehydrogenase and glycerol 3-phosphate phosphatase from S. cerevisiae was necessary to achieve high titers (Chen et al. Metabolic Engineering 2017 39:151-158), reportedly reaching ˜60 g/L of 3-HP in fed-batch fermentation.

Biosynthetic materials and methods, including organisms having increased production of 3-HP, derivatives thereof and compounds related thereto are needed.

### SUMMARY OF THE INVENTION

An aspect of the present invention relates to a process for biosynthesis of beta hydroxy acids, such as 3-HP including derivatives thereof and/or compounds related thereto. The process comprises obtaining an organism capable of producing 3-HP and derivatives and compounds related thereto, altering the organism, and producing more 3-HP and derivatives and compounds related thereto in the altered organism as compared to the unaltered organism. In one nonlimiting embodiment, the organism is C. necator or an organism with one or more properties similar thereto. In one nonlimiting embodiment, the organism is altered to express to express a glycerol dehydratase and/or a glycerol dehydratase reactivase and/or an aldehyde dehydrogenase and/or a glycerol 3-phosphate phosphatase and/or a glycerol 3-phosphate dehydrogenase.

In one nonlimiting embodiment, the glycerol dehydratase is from Klebsiella pneumoniae. In one nonlimiting embodiment, the glycerol dehydratase comprises SEQ ID NO:2, 5 and/or 7 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 2, 5 and/or 7 or a functional fragment thereof. In one nonlimiting embodiment, the glycerol dehydratase comprises a polypeptide encoded by a nucleic acid sequence of SEQ ID NO: 1 or 3 and/or 4 and/or 6, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by a nucleic acid sequence set forth in SEQ ID NO: 1 and 3 and/or 4 and/or 6 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 1 or 3 and/or 4 and/or 6 or a functional fragment thereof.

In one nonlimiting embodiment, the glycerol dehydratase reactivase is from Klebsiella pneumoniae. In one nonlimiting embodiment, the glycerol dehydratase reactivase comprises SEQ ID NO:9 and/or 10 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:9 and/or 10 or a functional fragment thereof. In one nonlimiting embodiment, the glycerol dehydratase reactivase comprises a polypeptide encoded by a nucleic acid sequence of SEQ ID NO: 8, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by the nucleic acid sequence set forth in SEQ ID NO: 8 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:8 or a functional fragment thereof.

In one nonlimiting embodiment, the aldehyde dehydrogenase is from Klebsiella pneumoniae or E. coli. In one nonlimiting embodiment, the aldehyde dehydrogenase comprises SEQ ID NO:12 or 14 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:12 or 14 or a functional fragment thereof. In one nonlimiting embodiment, the aldehyde dehydrogenase comprises a polypeptide encoded by a nucleic acid sequence of SEQ ID NO: 11 or 13, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by the nucleic acid sequence set forth in SEQ ID NO: 11 or 13 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:11 or SEQ ID NO:13 or a functional fragment thereof.

In one nonlimiting embodiment, the glycerol 3-phosphate phosphataseis GPP2 from S. cerevisiae. In one nonlimiting embodiment, the glycerol 3-phosphate phosphatase comprises SEQ ID NO:18 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:18 or a functional fragment thereof. In one nonlimiting embodiment, the glycerol 3-phosphate phosphatase comprises a polypeptide encoded by a nucleic acid sequence of SEQ ID NO: 17, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by the nucleic acid sequence set forth in SEQ ID NO: 17 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:17 or a functional fragment thereof.

In one nonlimiting embodiment, the glycerol 3-phosphate dehydrogenaseis GPD1 from S. cerevisiae. In one nonlimiting embodiment, the glycerol 3-phosphate dehydrogenase comprises SEQ ID NO:16 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:16 or a functional fragment thereof. In one nonlimiting embodiment, the glycerol 3-phosphate phosphatase comprises a polypeptide encoded by a nucleic acid sequence of SEQ ID NO: 15, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by the nucleic acid sequence set forth in SEQ ID NO: 15 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:15 or a functional fragment thereof.

In one nonlimiting embodiment, the nucleic acid sequence is codon optimized for C. necator.

In one nonlimiting embodiment, the organism is altered to express two or more of the enzymes of glycerol dehydratase, glycerol dehydratase reactivase, aldehyde dehydrogenase, glycerol 3-phosphate phosphatase and/or glycerol 3-phosphate dehydrogenase as disclosed herein.

In one nonlimiting embodiment, the organism is altered to express three or four of the enzymes of glycerol dehydratase, glycerol dehydratase reactivase, aldehyde dehydrogenase, glycerol 3-phosphate phosphatase and/or glycerol 3-phosphate dehydrogenase as disclosed herein.

In one nonlimiting embodiment, the organism is altered to express glycerol dehydratase, glycerol dehydratase reactivase, aldehyde dehydrogenase, glycerol 3-phosphate phosphatase and glycerol-3-phosphate dehydrogenase as disclosed herein.

In one nonlimiting embodiment, the organism is further altered to interfere with one or more genes involved in the degradation of 3-HP. In one nonlimiting embodiment, the gene is prpC1. In another nonlimiting embodiment the gene is mmsA1. In another nonlimiting embodiment the gene is mmsA2. In another nonlimiting embodiment the gene is mmsA3. In another nonlimiting embodiment the gene is hpdH. In another nonlimiting embodiment the gene is mmsB. In another nonlimiting embodiment, the gene encodes a glycerol kinase. In another nonlimiting embodiment, the gene encodes a CoA transferase or ligase. In another nonlimiting embodiment, one or more genes encoding one or more enzymes involved in converting 3-hydroxypropionate to succinyl-CoA are altered. In one nonlimiting embodiment, two or more of these genes are interfered with and/or more than one gene in a class of enzymes is interfered with.

In one nonlimiting embodiment, the organism is further modified to eliminate phaCAB, involved in PHBs production and/or H16-A0006-9 encoding endonucleases thereby improving transformation efficiency.

In one nonlimiting embodiment, the organism is altered to express, overexpress, not express or express less of one or more molecules depicted in FIG. 1A, 1B, 2 or 5. In one nonlimiting embodiment, the molecule(s) comprise a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence corresponding to a molecule(s) depicted in FIG. 1A, 1B, 2 or 5, or a functional fragment thereof.

Another aspect of the present invention relates to an organism altered to produce more 3-HP and/or derivatives and compounds related thereto as compared to the unaltered organism. In one nonlimiting embodiment, the organism is C. necator or an organism with properties similar thereto. In one nonlimiting embodiment, the organism is altered to express to express a glycerol dehydratase and/or a glycerol dehydratase reactivase and/or an aldehyde dehydrogenase.

In one nonlimiting embodiment, the glycerol dehydratase is from Klebsiella pneumoniae. In one nonlimiting embodiment, the glycerol dehydratase comprises SEQ ID NO:2, 5 and/or 7 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:2, 5 and/or 7 or a functional fragment thereof. In one nonlimiting embodiment, the glycerol dehydratase comprises a polypeptide encoded by a nucleic acid sequence of SEQ ID NO: 1 or 3 and/or 4 and/or 6, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by a nucleic acid sequence set forth in SEQ ID NO: 1 or 3 and/or 4 and/or 6 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 1 or 3 and/or 4 and/or 6 or a functional fragment thereof.

In one nonlimiting embodiment, the glycerol dehydratase reactivase is from Klebsiella pneumoniae. In one nonlimiting embodiment, the glycerol dehydratase reactivase comprises SEQ ID NO:9 and/or 10 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:9 and/or 10 or a functional fragment thereof. In one nonlimiting embodiment, the glycerol dehydratase reactivase comprises a polypeptide encoded by a nucleic acid sequence of SEQ ID NO: 8, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by the nucleic acid sequence set forth in SEQ ID NO: 8 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:8 or a functional fragment thereof.

In one nonlimiting embodiment, the aldehyde dehydrogenase is from Klebsiella pneumoniae or E. coli. In one nonlimiting embodiment, the aldehyde dehydrogenase comprises SEQ ID NO:12 or 14 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:12 or 14 or a functional fragment thereof. In one nonlimiting embodiment, the aldehyde dehydrogenase comprises a polypeptide encoded by a nucleic acid sequence of SEQ ID NO: 11 or 13, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by the nucleic acid sequence set forth in SEQ ID NO: 11 or 13 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:11 or SEQ ID NO:13 or a functional fragment thereof.

In one nonlimiting embodiment, the glycerol 3-phosphate phosphataseis GPP2 from S. cerevisiae. In one nonlimiting embodiment, the glycerol 3-phosphate phosphatase comprises SEQ ID NO:18 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:18 or a functional fragment thereof. In one nonlimiting embodiment, the glycerol 3-phosphate phosphatase comprises a polypeptide encoded by a nucleic acid sequence of SEQ ID NO: 17, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by the nucleic acid sequence set forth in SEQ ID NO: 17 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:17 or a functional fragment thereof. In one nonlimiting embodiment, the glycerol 3-phosphate dehydrogenase is GPD1 from S. cerevisiae. In one nonlimiting embodiment, the glycerol 3-phosphate dehydrogenase comprises SEQ ID NO:16 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:16 or a functional fragment thereof. In one nonlimiting embodiment, the glycerol 3-phosphate phosphatase comprises a polypeptide encoded by a nucleic acid sequence of SEQ ID NO:15, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by the nucleic acid sequence set forth in SEQ ID NO: 15 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:15 or a functional fragment thereof.

In one nonlimiting embodiment, the nucleic acid sequence is codon optimized for C. necator.

In one nonlimiting embodiment, the organism is further altered to interfere with one or more genes involved in the degradation of 3-HP. In one nonlimiting embodiment, the gene is prpC1. In another nonlimiting embodiment the gene is mmsA1. In another nonlimiting embodiment the gene is mmsA2. In another nonlimiting embodiment the gene is mmsA3. In another nonlimiting embodiment the gene is hpdH. In another nonlimiting embodiment the gene is mmsB. In another nonlimiting embodiment, the gene encodes a glycerol kinase. In another nonlimiting embodiment, the gene encodes a CoA transferase or ligase. In another nonlimiting embodiment, one or more genes encoding one or more enzymes involved in converting 3-hydroxypropionate to succinyl-CoA are altered. In one nonlimiting embodiment, two or more of these genes are interfered with and/or two more genes in a class are interfered with.

In one nonlimiting embodiment, the organism is further modified to eliminate phaCAB, involved in PHBs production and/or H16-A0006-9 encoding endonucleases thereby improving transformation efficiency.

Another aspect of the present invention relates to bio-derived, bio-based, or fermentation-derived products produced from any of the methods and/or altered organisms disclosed herein. Such products include compositions comprising at least one bio-derived, bio-based, or fermentation-derived compound or any combination thereof, as well as bio-derived, bio-based, or fermentation-derived polymers comprising these bio-derived, bio-based, or fermentation-derived compositions or compounds; bio-derived, bio-based, or fermentation-derived plastics comprising the bio-derived, bio-based, or fermentation-derived compositions or compounds or any combination thereof or the bio-derived, bio-based, or fermentation-derived plastics or any combination thereof; molded substances obtained by molding the bio-derived, bio-based, or fermentation-derived polymers or the bio-derived, bio-based, or fermentation-derived plastics or any combination thereof; bio-derived, bio-based, or fermentation-derived formulations comprising the bio-derived, bio-based, or fermentation-derived compositions or compounds, polymers or plastics, or the bio-derived, bio-based, or fermentation-derived molded substances, or any combination thereof; and bio-derived, bio-based, or fermentation-derived semi-solids or non-semi-solid streams comprising the bio-derived, bio-based, or fermentation-derived compositions or compounds, polymers, plastics, molded substances or formulations, or any combination thereof.

Another aspect of the present invention relates to a bio-derived, bio-based or fermentation derived product biosynthesized in accordance with the exemplary central metabolism depicted in FIGS. 1A, 1B, 2 and 5.

Another aspect of the present invention relates to exogenous genetic molecules of the altered organisms disclosed herein. In one nonlimiting embodiment, the exogenous genetic molecule comprises a codon optimized nucleic acid sequence encoding a glycerol dehydratase, a glycerol dehydratase reactivase, glycerol-3-phosphate dehydrogenase and/or an aldehyde dehydrogenase and/or glycerol 3-phosphate phosphatase. In one nonlimiting embodiment, the nucleic acid sequence is codon optimized for C. necator. In one nonlimiting embodiment, the exogenous genetic molecule comprises a nucleic acid sequence encoding Klebsiella pneumoniae glycerol dehydratase. In one nonlimiting embodiment, the exogenous genetic molecule comprises SEQ ID NO: 1 or 3 and/or 4 and/or 6, a nucleic acid sequence exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 1 or 3 and/or 4 and/or 6 or a functional fragment thereof, or a nucleic acid sequence encoding a polypeptide with similar enzymatic activities and at least about 50%, 60%, 70%, 75%, 80%, 85%, 90%, 91%, 92%, 93%, 94%, 95%, 96%, 97%, 98%, 99% or 99.5% sequence identity to the polypeptide encoded by the nucleic acid sequence set forth in SEQ ID NO: 1 or 3 and/or 4 and/or 6 or a functional fragment thereof. In one nonlimiting embodiment, the exogenous genetic molecule comprises a nucleic acid sequence encoding Klebsiella pneumoniae glycerol dehydratase reactivase. In one nonlimiting embodiment, the exogenous genetic molecule comprises SEQ ID NO: 8, a nucleic acid sequence exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 8 or a functional fragment thereof, or a nucleic acid sequence encoding a polypeptide with similar enzymatic activities and at least about 50%, 60%, 70%, 75%, 80%, 85%, 90%, 91%, 92%, 93%, 94%, 95%, 96%, 97%, 98%, 99% or 99.5% sequence identity to the polypeptide encoded by the nucleic acid sequence set forth in SEQ ID NO:8 or a functional fragment thereof. In one nonlimiting embodiment, the exogenous genetic molecule comprises a nucleic acid sequence encoding an aldehyde dehydrogenase from Klebsiella pneumoniae or E. coli. In one nonlimiting embodiment, the exogenous genetic molecule comprises SEQ ID NO: 11 or 13, a nucleic acid sequence exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 11 or 13 or a functional fragment thereof, or a nucleic acid sequence encoding a polypeptide with similar enzymatic activities and at least about 50%, 60%, 70%, 75%, 80%, 85%, 90%, 91%, 92%, 93%, 94%, 95%, 96%, 97%, 98%, 99% or 99.5% sequence identity to the polypeptide encoded by SEQ ID NO:11 or 13 or a functional fragment thereof. In one nonlimiting embodiment, the exogenous genetic molecule comprises a nucleic acid sequence encoding a glycerol 3-phosphate phosphatase from S. cerevisiae. In one nonlimiting embodiment, the exogenous genetic molecule comprises a nucleic acid sequence encoding SEQ ID NO: 17, a nucleic acid sequence exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 17 or a functional fragment thereof, or a nucleic acid sequence encoding a polypeptide with similar enzymatic activities and at least about 50%, 60%, 70%, 75%, 80%, 85%, 90%, 91%, 92%, 93%, 94%, 95%, 96%, 97%, 98%, 99% or 99.5% sequence identity to the polypeptide encoded by SEQ ID NO:17 or a functional fragment thereof. In one nonlimiting embodiment, the exogenous genetic molecule comprises a nucleic acid sequence encoding a glycerol 3-phosphate dehydrogenase from S. cerevisiae. In one nonlimiting embodiment, the exogenous genetic molecule comprises a nucleic acid sequence encoding SEQ ID NO: 15, a nucleic acid sequence exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 15 or a functional fragment thereof, or a nucleic acid sequence encoding a polypeptide with similar enzymatic activities and at least about 50%, 60%, 70%, 75%, 80%, 85%, 90%, 91%, 92%, 93%, 94%, 95%, 96%, 97%, 98%, 99% or 99.5% sequence identity to the polypeptide encoded by SEQ ID NO:15 or a functional fragment thereof. Additional nonlimiting examples of exogenous genetic molecules include expression constructs of, for example, a glycerol dehydratase and/or a glycerol dehydratase reactivase and/or an aldehyde dehydrogenase and/or a glycerol 3-phosphate phosphatase and/or a glycerol 3-phosphate dehydrogenase and synthetic operons of, for example a glycerol dehydratase and/or a glycerol dehydratase reactivase and/or an aldehyde dehydrogenase and/or a glycerol 3-phosphate phosphatase and/or a glycerol 3-phosphate dehydrogenase.

Yet another aspect of the present invention relates to means and processes for use of these means for biosynthesis of beta hydroxy acids, such as 3-HP including derivatives thereof and/or compounds related thereto.

### BRIEF DESCRIPTION OF THE FIGURES

FIG. 1A is a schematic representation of the 3-HP pathway from glycerol. GDH: glycerol dehydratase classified in EC 4.2.1.30; Co-B12: vitamin B12; ALDH: aldehyde dehydrogenase classified in EC 1.1.1.8, EC 1.2.1.3 or EC 1.2.1.86.

FIG. 1B is a schematic representation of the 3-HP pathway from fructose.

FIG. 2 is a schematic representation of the pathway for glycerol synthesis from fructose. frk: fructokinase; pgi: glucose-6-phosphate isomerase; zwf: glucose 6-phosphate 1-dehydrogenase; pgl: 6-phosphogluconolactonase; edd: phosphogluconate dehydratase; eda: 2-keto-3-deoxy-6-phosphogluconate aldolase; tpi: triosephosphate isomerase; gpd: glycerol 3-phosphate dehydrogenase as, for example, classified in EC 1.1.1.8; gpp: glycerol 3-phosphate phosphatase as, for example, classified in EC 3.1.3.21 (not been described in C. necator).

FIG. 3 is a schematic representation of the distribution of the mmsA genes, hpdH and mmsB in the genome of C. necator. Chromosome 1 includes the mmsA1 gene, the operon composed of the regulatory gene hpdR (LysR-TR), and genes mmsA2 and hpdH. Chromosome 2 includes the operon composed of the regulatory gene araC, and genes araD, mmsA3 and mmsB.

FIG. 4A is a schematic representation of the distribution of genes dhaB123, gdrAB, and aldH or puuC in the expression vector pBBR1-1A.

FIG. 4B is a schematic representation of the distribution of genes GPD1, and GPP2 in the expression vector pMOL28-2A.

FIG. 5 is a schematic representation of the oxidative and reductive routes for the degradation of 3-hydroxypropionate.

### DETAILED DESCRIPTION

The present invention provides processes for biosynthesis of beta hydroxy acids, such as 3-hydroxypropanoic acid (3-HP) and/or derivatives thereof, and/or compounds related thereto, and organisms altered to increase biosynthesis of 3-HP, derivatives thereof and compounds related thereto, and organisms related thereto, exogenous genetic molecules of these altered organisms, and bio-derived, bio-based, or fermentation-derived products biosynthesized or otherwise produced by any of these methods and/or altered organisms.

In one aspect of the present invention, the carbon flux of the fructose biochemical node in an organism is redirected to produce 3-HP by alteration of the organism to express a glycerol dehydratase and/or a glycerol dehydratase reactivase and/or an aldehyde dehydrogenase and/or a glycerol 3-phosphate phosphatase and/or a glycerol 3-phosphate dehydrogenase. Organisms produced in accordance with the present invention are useful in methods for biosynthesizing higher levels of 3-HP, derivatives thereof, and compounds related thereto.

For purposes of the present invention, by “3-hydroxypropanoic acid (3-HP)” it is meant to encompass 3-hydroxypropanate and other C2 and C3 acids.

For purposes of the present invention, by “derivatives and compounds related thereto” it is meant to encompass compounds derived from the same substrates and/or enzymatic reactions as compounds involved in 3-HP metabolism, byproducts of these enzymatic reactions and compounds with similar chemical structure including, but not limited to, structural analogs wherein one or more substituents of compounds involved in 3-HP metabolism are replaced with alternative substituents. Nonlimiting examples include 2-propen-1-ol, propanedioic acid, 1,3-propanediol and propanedial. As will be understood by the skilled artisan, this list is in no way exhaustive.

For purposes of the present invention, by “higher levels of 3-HP” it is meant that the altered organisms and methods of the present invention are capable of producing increased levels of 3-HP and derivatives and compounds related thereto as compared to the same organism without alteration.

For compounds containing carboxylic acid groups such as organic monoacids, hydroxyacids, amino acids and dicarboxylic acids, these compounds may be formed or converted to their ionic salt form when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate and/or bicarbonate, sodium hydroxide, ammonia and the like. The salt can be isolated as is from the system as the salt or converted to the free acid by reducing the pH to, for example, below the lowest pKa through addition of acid or treatment with an acidic ion exchange resin.

For compounds containing amine groups such as, but not limited to, organic amines, amino acids and diamine, these compounds may be formed or converted to their ionic salt form by addition of an acidic proton to the amine to form the ammonium salt, formed with inorganic acids such as hydrochloric acid, hydrobromic acid, sulfuric acid, nitric acid, phosphoric acid, and the like; or formed with organic acids such as carbonic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid or muconic acid, and the like. The salt can be isolated as is from the system as a salt or converted to the free amine by raising the pH to, for example, above the highest pKa through addition of base or treatment with a basic ion exchange resin. Acceptable inorganic bases are known in the art and include aluminum hydroxide, calcium hydroxide, potassium hydroxide, sodium carbonate or bicarbonate, sodium hydroxide, and the like.

For compounds containing both amine groups and carboxylic acid groups such as, but not limited to, amino acids, these compounds may be formed or converted to their ionic salt form by either 1) acid addition salts, formed with inorganic acids such as hydrochloric acid, hydrobromic acid, sulfuric acid, nitric acid, phosphoric acid, and the like; or formed with organic acids such as carbonic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. Acceptable inorganic bases include aluminum hydroxide, calcium hydroxide, potassium hydroxide, sodium carbonate and/or bicarbonate, sodium hydroxide, and the like, or 2) when an acidic proton present in the parent compound either is replaced by a metal ion, e.g., an alkali metal ion, an alkaline earth ion, or an aluminum ion; or coordinates with an organic base. Acceptable organic bases are known in the art and include ethanolamine, diethanolamine, triethanolamine, trimethylamine, N-methylglucamine, and the like. Acceptable inorganic bases are known in the art and include aluminum hydroxide, calcium hydroxide, potassium hydroxide, sodium carbonate, sodium hydroxide, ammonia and the like. The salt can be isolated as is from the system or converted to the free acid by reducing the pH to, for example, below the pKa through addition of acid or treatment with an acidic ion exchange resin. In one or more aspects of the invention, it is understood that the amino acid salt can be isolated as: i. at low pH, as the ammonium (salt)-free acid form; ii. at high pH, as the amine-carboxylic acid salt form; and/or iii. at neutral or midrange pH, as the free-amine acid form or zwitterion form.

In the process for biosynthesis of 3-HP and derivatives and compounds related thereto of the present invention, an organism capable of producing 3-HP and derivatives and compounds related thereto is obtained. The organism is then altered to produce more 3-HP and derivatives and compounds related thereto in the altered organism as compared to the unaltered organism.

In one nonlimiting embodiment, the organism is Cupriavidus necator (C. necator) or an organism with properties similar thereto. A nonlimiting embodiment of the organism is set for at lgcstandards-atcc with the extension .org/products/all/17699.aspx?geo_country=gb#generalinformation of the world wide web.

C. necator (previously called Hydrogenomonas eutrophus, Alcaligenes eutropha, Ralstonia eutropha, and Wautersia eutropha) is a Gram-negative, flagellated soil bacterium of the Betaproteobacteria class. This hydrogen-oxidizing bacterium is capable of growing at the interface of anaerobic and aerobic environments and easily adapts between heterotrophic and autotrophic lifestyles. Sources of energy for the bacterium include both organic compounds and hydrogen. C. necator does not naturally contain genes for RCM and therefore does not express this enzyme. Additional properties of C. necator include microaerophilicity, copper resistance (Makar, N. S. & Casida, L. E. Int. J. of Systematic Bacteriology 1987 37(4): 323-326), bacterial predation (Byrd et al. Can J Microbiol 1985 31:1157-1163; Sillman, C. E. & Casida, L. E. Can J Microbiol 1986 32:760-762; Zeph, L. E. & Casida, L. E. Applied and Environmental Microbiology 1986 52(4):819-823) and polyhydroxybutyrate (PHB) synthesis. In addition, the cells have been reported to be capable of both aerobic and nitrate dependent anaerobic growth. A nonlimiting example of a C. necator organism useful in the present invention is a C. necator of the H16 strain. In one nonlimiting embodiment, a C. necator host of the H16 strain with at least a portion of the phaCAB gene locus knocked out (ΔphaCAB) is used.

In another nonlimiting embodiment, the organism altered in the process of the present invention has one or more of the above-mentioned properties of Cupriavidus necator.

In another nonlimiting embodiment, the organism is selected from members of the genera Ralstonia, Wautersia, Cupriavidus, Alcaligenes, Burkholderia or Pandoraea.

Cupriavidus necator lacks a phosphofructokinase enzyme that catalyzes the conversion of fructose 6-phosphate to fructose 1,6-bisphosphate in the Embden-Meyerhof-Parnas pathway. This organism metabolizes hexoses to glyceraldehyde 3-phosphate by the Entner-Doudoroff pathway (Chen et al. PNAS 2016 113(19):5441-5446). Then, glyceraldehyde 3-phosphate enters the glycolytic pathway where it is metabolized to pyruvate. It can also be isomerized to dihydroxyacetone phosphate by a triose phosphate isomerase, then converted into glycerol 3-phosphate by the action of glycerol 3-phosphate dehydrogenase and be used in the synthesis of glycerolipids. In some organisms, like yeast, glycerol can be produced from glycerol 3-phosphate in a reaction catalyzed by glycerol 3-phosphate phosphatase. While this specific enzyme is not present in C. necator, its action could be replaced by non-specific enzymes in this organism. A degradation pathway specific for 3-hydroxypropionate has been described in Pseudomonas denitrificans (Zhou et al. Biotechnology for Biofuels 2015 8:169). In this organism, 3-HP is converted into malonate semialdehyde and then into acetyl-CoA by the action of two enzymes encoded by hpdH and mmsA. These genes have been identified in C. necator by homology. Accordingly, this degradation pathway appears to be present in this organism. Therefore, interference with the genes involved may be necessary in order to accumulate this compound.

3-HP can be also assimilated by the methylcitrate cycle. In this case, 3-HP is converted to propyonyl-CoA, with 3-hydroxypropionyl-CoA and acryloyl-CoA as intermediates, before entering in this cycle. A propionate CoA transferase with in vitro specificity for 3-HP has been described in C. necator (Lindenkamp et al. Appl Microbiol Biotechnol 2013 97:7699-7709; Volodina et al. Appl Microbiol Biotechnol 2014 98:3579-3589), so degradation of this compound through this pathway is also possible.

Accordingly, for the process of the present invention, the organism is altered to express a glycerol dehydratase and/or a glycerol dehydratase reactivase and/or an aldehyde dehydrogenase and/or a glycerol 3-phosphate phosphatase and/or a glycerol 3-phosphate dehydrogenase.

In one nonlimiting embodiment, the organism is altered to express a glycerol dehydratase. In one nonlimiting embodiment, the glycerol dehydratase is from Klebsiella pneumoniae. In one nonlimiting embodiment, the glycerol dehydratase comprises SEQ ID NO:2, 5 and/or 7 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:2, 5 and/or 7 or a functional fragment thereof. In one nonlimiting embodiment, the glycerol dehydratase comprises a polypeptide encoded by a nucleic acid sequence of SEQ ID NO: 1 or 3 and/or 4 and/or 6, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by a nucleic acid sequence set forth in SEQ ID NO: 1 or 3 and/or 4 and/or 6 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 1 or 3 and/or 4 and/or 6 or a functional fragment thereof. In one nonlimiting embodiment, the glycerol dehydratase enzyme is classified in EC 4.2.1.30.

In another nonlimiting embodiment, the organism is altered to express a glycerol dehydratase reactivase. In one nonlimiting embodiment, the glycerol dehydratase reactivase is from Klebsiella pneumoniae. In one nonlimiting embodiment, the glycerol dehydratase reactivase comprises SEQ ID NO:9 and/or 10 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:9 and/or 10 or a functional fragment thereof. In one nonlimiting embodiment, the glycerol dehydratase reactivase comprises a polypeptide encoded by a nucleic acid sequence of SEQ ID NO: 8, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by the nucleic acid sequence set forth in SEQ ID NO: 8 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:8 or a functional fragment thereof.

In another nonlimiting embodiment, the organism is altered to express aldehyde dehydrogenase. In one nonlimiting embodiment, the aldehyde dehydrogenase is from Klebsiella pneumoniae or E. coli. In one nonlimiting embodiment, the aldehyde dehydrogenase comprises SEQ ID NO:12 or 14 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:12 or 14 or a functional fragment thereof. In one nonlimiting embodiment, the aldehyde dehydrogenase comprises a polypeptide encoded by a nucleic acid sequence of SEQ ID NO: 11 or 13, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by the nucleic acid sequence set forth in SEQ ID NO: 11 or 13 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:11 or SEQ ID NO:13 or a functional fragment thereof.

In one nonlimiting embodiment, the dehydrogenase enzyme is classified in EC 1.1.1.8, EC 1.2.1.3 or EC 1.2.1.B6.

In one nonlimiting embodiment, the organism is altered to express glycerol 3-phosphate phosphatase. In one nonlimiting embodiment, the glycerol 3-phosphate phosphatase is GPP2 from S. cerevisiae. In one nonlimiting embodiment, the glycerol 3-phosphate phosphatase comprises SEQ ID NO:18 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:18 or a functional fragment thereof. In one nonlimiting embodiment, the glycerol 3-phosphate phosphatase comprises a polypeptide encoded by a nucleic acid sequence of SEQ ID NO: 17, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by the nucleic acid sequence set forth in SEQ ID NO: 17 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:17 or a functional fragment thereof.

In one nonlimiting embodiment, the glycerol 3-phosphate dehydrogenaseis GPD1 from S. cerevisiae. In one nonlimiting embodiment, the glycerol 3-phosphate dehydrogenase comprises SEQ ID NO:16 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:16 or a functional fragment thereof. In one nonlimiting embodiment, the glycerol 3-phosphate phosphatase comprises a polypeptide encoded by a nucleic acid sequence of SEQ ID NO: 15, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by the nucleic acid sequence set forth in SEQ ID NO: 15 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:15 or a functional fragment thereof. In one nonlimiting embodiment, the nucleic acid sequence is codon optimized for C. necator.

In one nonlimiting embodiment, the organism is altered to express two or more of the enzymes of glycerol dehydratase, glycerol dehydratase reactivase, aldehyde dehydrogenase, glycerol 3-phosphate phosphatase and/or glycerol 3-phosphate dehydrogenase as disclosed herein.

In one nonlimiting embodiment, the organism is altered to express three or more of the enzymes of glycerol dehydratase, glycerol dehydratase reactivase, aldehyde dehydrogenase, glycerol 3-phosphate phosphatase and/or glycerol 3-phosphate dehydrogenase as disclosed herein.

In one nonlimiting embodiment, the organism is altered to express four or more of the enzymes of glycerol dehydratase, glycerol dehydratase reactivase, aldehyde dehydrogenase, glycerol 3-phosphate phosphatase and/or glycerol 3-phosphate dehydrogenase as disclosed herein.

In one nonlimiting embodiment, the organism is altered to express glycerol dehydratase, glycerol dehydratase reactivase, aldehyde dehydrogenase, glycerol 3-phosphate phosphatase and/or glycerol 3-phosphate dehydrogenase as disclosed herein.

In one nonlimiting embodiment, the organism is further altered to interfere with one or more genes involved in the degradation of 3-HP. In one nonlimiting embodiment, the gene is prpC1. In another nonlimiting embodiment the gene is mmsA1. In another nonlimiting embodiment the gene is mmsA2. In another nonlimiting embodiment the gene is mmsA3. In another nonlimiting embodiment the gene is hpdH. In another nonlimiting embodiment the gene is mmsB. In another nonlimiting embodiment, the gene encodes a glycerol kinase. In another nonlimiting embodiment, the gene encodes a CoA transferase or ligase. In another nonlimiting embodiment, one or more genes encoding one or more enzymes involved in converting 3-hydroxypropionate to succinyl-CoA are altered. In one nonlimiting embodiment, two or more of these genes are interfered with and/or two more genes in a class are interfered with.

As used herein, by “interference with” or “interfered with” it is meant to encompass any physical or chemical change to the organism which ultimately decreases activity of the enzyme. Examples include, but are in no way limited to, mutation or deletion of a gene encoding the enzyme, addition of an enzyme inhibitor and addition of an agent which decreases or inhibits expression of the enzyme.

In one nonlimiting embodiment, the organism is further modified to eliminate phaCAB, involved in PHBs production and/or H16-A0006-9 encoding endonucleases thereby improving transformation efficiency as described in U.S. patent application Ser. No. 15/717,216, teachings of which are incorporated herein by reference.

In the process of the present invention, the altered organism is then subjected to conditions wherein 3-HP and derivatives and compounds related thereto are produced. In the process described herein, a fermentation strategy can be used that entails anaerobic, micro-aerobic or aerobic cultivation. A fermentation strategy can entail nutrient limitation such as nitrogen, phosphate or oxygen limitation.

Under conditions of nutrient limitation a phenomenon known as overflow metabolism (also known as energy spilling, uncoupling or spillage) occurs in many bacteria (Russell, 2007). In growth conditions in which there is a relative excess of carbon source and other nutrients (e.g. phosphorous, nitrogen and/or oxygen) are limiting cell growth, overflow metabolism results in the use of this excess energy (or carbon), not for biomass formation but for the excretion of metabolites, typically organic acids. In Cupriavidus necator a modified form of overflow metabolism occurs in which excess carbon is sunk intracellularly into the storage carbohydrate polyhydroxybutyrate (PHB). In strains of C. necator which are deficient in PHB synthesis this overflow metabolism can result in the production of extracellular overflow metabolites. The range of metabolites that have been detected in PHB deficient C. necator strains include acetate, acetone, butanoate, cis-aconitate, citrate, ethanol, fumarate, 3-hydroxybutanoate, propan-2-ol, malate, methanol, 2-methyl-propanoate, 2-methyl-butanoate, 3-methyl-butanoate, 2-oxoglutarate, meso-2,3-butanediol, acetoin, DL-2,3-butanediol, 2-methylpropan-1-ol, propan-1-ol, lactate 2-oxo-3-methylbutanoate, 2-oxo-3-methylpentanoate, propanoate, succinate, formic acid and pyruvate. The range of overflow metabolites produced in a particular fermentation can depend upon the limitation applied (e.g. nitrogen, phosphate, oxygen), the extent of the limitation, and the carbon source provided (Schlegel, H. G. & Vollbrecht, D. Journal of General Microbiology 1980 117:475-481; Steinbüchel, A. & Schlegel, H. G. Appl Microbiol Biotechnol 1989 31: 168; Vollbrecht et al. Eur J Appl Microbiol Biotechnol 1978 6:145-155; Vollbrecht et al. European J. Appl. Microbiol. Biotechnol. 1979 7: 267; Vollbrecht, D. & Schlegel, H. G. European J. Appl. Microbiol. Biotechnol. 1978 6: 157; Vollbrecht, D. & Schlegel, H. G. European J. Appl. Microbiol. Biotechnol. 1979 7: 259).

Applying a suitable nutrient limitation in defined fermentation conditions can thus result in an increase in the flux through a particular metabolic node. The application of this knowledge to C. necator strains genetically modified to produce desired chemical products via the same metabolic node can result in increased production of the desired product.

A cell retention strategy using a ceramic hollow fiber membrane can be employed to achieve and maintain a high cell density during fermentation. The principal carbon source fed to the fermentation can derive from a biological or non-biological feedstock. The biological feedstock can be, or can derive from, monosaccharides, disaccharides, lignocellulose, hemicellulose, cellulose, paper-pulp waste, black liquor, lignin, levulinic acid and formic acid, triglycerides, glycerol, fatty acids, agricultural waste, thin stillage, condensed distillers' solubles or municipal waste such as fruit peel/pulp. The non-biological feedstock can be, or can derive from, natural gas, syngas, CO₂/H₂, CO, H₂, O₂, methanol, ethanol, non-volatile residue (NVR) a caustic wash waste stream from cyclohexane oxidation processes or waste stream from a chemical industry such as, but not limited to a carbon black industry or a hydrogen-refining industry, or petrochemical industry, a nonlimiting example being a PTA-waste stream.

In one nonlimiting embodiment, at least one of the enzymatic conversions of the 3-HP production method comprises gas fermentation within the altered Cupriavidus necator host, or a member of the genera Ralstonia, Wautersia, Alcaligenes, Burkholderia and Pandoraea, and other organism having one or more of the above-mentioned properties of Cupriavidus necator. In this embodiment, the gas fermentation may comprise at least one of natural gas, syngas, CO₂/H₂, CO, H₂, O₂, methanol, ethanol, non-volatile residue, caustic wash from cyclohexane oxidation processes, or waste stream from a chemical industry such as, but not limited to a carbon black industry or a hydrogen-refining industry, or petrochemical industry. In one nonlimiting embodiment, the gas fermentation comprises CO₂/H₂.

The methods of the present invention may further comprise recovering produced 3-HP or derivatives or compounds related thereto. Once produced, any method can be used to isolate the 3-HP or derivatives or compounds related thereto.

The present invention also provides altered organisms capable of biosynthesizing increased amounts of 3-HP and derivatives and compounds related thereto as compared to the unaltered organism. In one nonlimiting embodiment, the altered organism of the present invention is a genetically engineered strain of Cupriavidus necator capable of producing 3-HP and derivatives and compounds related thereto. In another nonlimiting embodiment, the organism to be altered is selected from members of the genera Ralstonia, Wautersia, Alcaligenes, Cupriavidus, Burkholderia and Pandoraea, and other organisms having one or more of the above-mentioned properties of Cupriavidus necator. In one nonlimiting embodiment, the present invention relates to a substantially pure culture of the altered organism capable of producing 3-HP and derivatives and compounds related thereto via a glycerol dehydratase and/or a glycerol dehydratase reactivase and/or an aldehyde dehydrogenase and/or a glycerol 3-phosphate phosphatase and/or a glycerol 3-phosphate dehydrogenase pathway.

As used herein, a “substantially pure culture” of an altered organism is a culture of that microorganism in which less than about 40% (i.e., less than about 35%; 30%; 25%; 20%; 15%; 10%; 5%; 2%; 1%; 0.5%; 0.25%; 0.1%; 0.01%; 0.001%; 0.0001%; or even less) of the total number of viable cells in the culture are viable cells other than the altered microorganism, e.g., bacterial, fungal (including yeast), mycoplasmal, or protozoan cells. The term “about” in this context means that the relevant percentage can be 15% of the specified percentage above or below the specified percentage. Thus, for example, about 20% can be 17% to 23%. Such a culture of altered microorganisms includes the cells and a growth, storage, or transport medium. Media can be liquid, semi-solid (e.g., gelatinous media), or frozen. The culture includes the cells growing in the liquid or in/on the semi-solid medium or being stored or transported in a storage or transport medium, including a frozen storage or transport medium. The cultures are in a culture vessel or storage vessel or substrate (e.g., a culture dish, flask, or tube or a storage vial or tube).

Altered organisms of the present invention comprise at least one genome-integrated synthetic operon encoding an enzyme.

In one nonlimiting embodiment, the altered organism is produced by integration of a synthetic operon encoding a glycerol dehydratase and/or a glycerol dehydratase reactivase and/or an aldehyde dehydrogenase and/or a glycerol 3-phosphate phosphatase and/or a glycerol 3-phosphate dehydrogenase.

In one nonlimiting embodiment, the glycerol dehydratase is from Klebsiella pneumoniae. In one nonlimiting embodiment, the glycerol dehydratase comprises SEQ ID NO:2, 5 and/or 7 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:2, 5 and/or 7 or a functional fragment thereof. In one nonlimiting embodiment, the glycerol dehydratase comprises a polypeptide encoded by a nucleic acid sequence of SEQ ID NO: 1 or 3 and/or 4 and/or 6, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by a nucleic acid sequence set forth in SEQ ID NO:1 or 3 and/or 4 and/or 6 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 1 or 3 and/or 4 and/or 6 or a functional fragment thereof. In one nonlimiting embodiment, the glycerol dehydratase enzyme is classified in EC 4.2.1.30.

In another nonlimiting embodiment, the glycerol dehydratase reactivase is from Klebsiella pneumoniae. In one nonlimiting embodiment, the glycerol dehydratase reactivase comprises SEQ ID NO:9 and/or 10 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:9 and/or 10 or a functional fragment thereof. In one nonlimiting embodiment, the glycerol dehydratase reactivase comprises a polypeptide encoded by a nucleic acid sequence of SEQ ID NO: 8, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by the nucleic acid sequence set forth in SEQ ID NO: 8 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:8 or a functional fragment thereof.

In another nonlimiting embodiment, the aldehyde dehydrogenase is from Klebsiella pneumoniae or E. coli. In one nonlimiting embodiment, the aldehyde dehydrogenase comprises SEQ ID NO:12 or 14 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:12 or 14 or a functional fragment thereof. In one nonlimiting embodiment, the aldehyde dehydrogenase comprises a polypeptide encoded by a nucleic acid sequence of SEQ ID NO: 11 or 13, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by the nucleic acid sequence set forth in SEQ ID NO: 11 or 13 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:11 or SEQ ID NO:13 or a functional fragment thereof.

In one nonlimiting embodiment, the dehydrogenase enzyme is classified in EC 1.1.1.8, EC 1.2.1.3 or EC 1.2.1.B6.

In one nonlimiting embodiment, the organism is altered to express glycerol 3-phosphate phosphatase. In one nonlimiting embodiment, the glycerol 3-phosphate phosphatase is GPP2 from S. cerevisiae. In one nonlimiting embodiment, the glycerol 3-phosphate phosphatase comprises SEQ ID NO:18 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:18 or a functional fragment thereof. In one nonlimiting embodiment, the glycerol 3-phosphate phosphatase comprises a polypeptide encoded by a nucleic acid sequence of SEQ ID NO: 17, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by the nucleic acid sequence set forth in SEQ ID NO: 17 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:17 or a functional fragment thereof.

In one nonlimiting embodiment, the glycerol 3-phosphate dehydrogenaseis GPD1 from S. cerevisiae. In one nonlimiting embodiment, the glycerol 3-phosphate dehydrogenase comprises SEQ ID NO:16 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:16 or a functional fragment thereof. In one nonlimiting embodiment, the glycerol 3-phosphate phosphatase comprises a polypeptide encoded by a nucleic acid sequence of SEQ ID NO: 15, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by the nucleic acid sequence set forth in SEQ ID NO: 15 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:15 or a functional fragment thereof.

In one nonlimiting embodiment, the nucleic acid sequence is codon optimized for C. necator.

In one nonlimiting embodiment, the organism is altered to express two or more of the enzymes of glycerol dehydratase, glycerol dehydratase reactivase, aldehyde dehydrogenase, glycerol 3-phosphate phosphatase and/or glycerol 3-phosphate dehydrogenase as disclosed herein.

In one nonlimiting embodiment, the organism is altered to express three or more of the enzymes of glycerol dehydratase, glycerol dehydratase reactivase, aldehyde dehydrogenase, glycerol 3-phosphate phosphatase and/or glycerol 3-phosphate dehydrogenase as disclosed herein.

In one nonlimiting embodiment, the organism is altered to express four or more of the enzymes of glycerol dehydratase, glycerol dehydratase reactivase, aldehyde dehydrogenase, glycerol 3-phosphate phosphatase and/or glycerol 3-phosphate dehydrogenase as disclosed herein.

In one nonlimiting embodiment, the organism is altered to express glycerol dehydratase, glycerol dehydratase reactivase, aldehyde dehydrogenase, glycerol 3-phosphate phosphatase and glycerol 3-phosphate dehydrogenase as disclosed herein.

In one nonlimiting embodiment, the organism is further altered to interfered with one or more genes involved in the degradation of 3-HP. In one nonlimiting embodiment, the gene is prpC1. In another nonlimiting embodiment the gene is mmsA1. In another nonlimiting embodiment the gene is mmsA2. In another nonlimiting embodiment the gene is mmsA3. In another nonlimiting embodiment the gene is hpdH. In another nonlimiting embodiment the gene is mmsB. In another nonlimiting embodiment, the gene encodes a glycerol kinase. In another nonlimiting embodiment, the gene encodes a CoA transferase or ligase. In another nonlimiting embodiment, one or more genes encoding one or more enzymes involved in converting 3-hydroxypropionate to succinyl-CoA are altered. In one nonlimiting embodiment, two or more of these genes are interfered with and/or more than one gene in a class of enzymes is interfered with.

In one nonlimiting embodiment, the organism is further modified to eliminate phaCAB, involved in PHBs production and/or H16-A0006-9 encoding endonucleases thereby improving transformation efficiency.

The percent identity (and/or homology) between two amino acid sequences as disclosed herein can be determined as follows. First, the amino acid sequences are aligned using the BLAST 2 Sequences (B12seq) program from the stand-alone version of BLAST containing BLASTP version 2.0.14. This stand-alone version of BLAST can be obtained from the U.S. government's National Center for Biotechnology Information web site (www with the extension ncbi.nlm.nih.gov). Instructions explaining how to use the B12seq program can be found in the readme file accompanying BLASTZ. B12seq performs a comparison between two amino acid sequences using the BLASTP algorithm. To compare two amino acid sequences, the options of B12seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq-i c:\seq1.txt-j c:\seq2.txt-p blastp-o c:\output.txt. If the two compared sequences share homology (identity), then the designated output file will present those regions of homology as aligned sequences. If the two compared sequences do not share homology (identity), then the designated output file will not present aligned sequences. Similar procedures can be followed for nucleic acid sequences except that blastn is used.

Once aligned, the number of matches is determined by counting the number of positions where an identical amino acid residue is presented in both sequences. The percent identity (homology) is determined by dividing the number of matches by the length of the full-length polypeptide amino acid sequence followed by multiplying the resulting value by 100. It is noted that the percent identity (homology) value is rounded to the nearest tenth. For example, 90.11, 90.12, 90.13, and 90.14 is rounded down to 90.1, while 90.15, 90.16, 90.17, 90.18, and 90.19 is rounded up to 90.2. It also is noted that the length value will always be an integer.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given enzyme can be modified such that optimal expression in a particular species (e.g., bacteria or fungus) is obtained, using appropriate codon bias tables for that species.

Functional fragments of any of the polypeptides or nucleic acid sequences described herein can also be used in the methods and organisms disclosed herein. The term “functional fragment” as used herein refers to a peptide fragment of a polypeptide or a nucleic acid sequence fragment encoding a peptide fragment of a polypeptide that has at least about 25% (e.g., at least about 30%; 40%; 50%; 60%; 70%; 75%; 80%; 85%; 90%; 95%; 98%; 99%; 100%; or even greater than 100%) of the activity of the corresponding mature, full-length, polypeptide. The functional fragment can generally, but not always, be comprised of a continuous region of the polypeptide, wherein the region has functional activity.

Functional fragments may range in length from about 10% up to 99% (inclusive of all percentages in between) of the original full-length sequence.

This document also provides (i) functional variants of the enzymes used in the methods of the document and (ii) functional variants of the functional fragments described above. Functional variants of the enzymes and functional fragments can contain additions, deletions, or substitutions relative to the corresponding wild-type sequences. Enzymes with substitutions will generally have not more than 50 (e.g., not more than one, two, three, four, five, six, seven, eight, nine, ten, 12, 15, 20, 25, 30, 35, 40, or 50) amino acid substitutions (e.g., conservative substitutions). This applies to any of the enzymes described herein and functional fragments. A conservative substitution is a substitution of one amino acid for another with similar characteristics. Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The nonpolar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above-mentioned polar, basic or acidic groups by another member of the same group can be deemed a conservative substitution. By contrast, a nonconservative substitution is a substitution of one amino acid for another with dissimilar characteristics.

Deletion variants can lack one, two, three, four, five, six, seven, eight, nine, ten, 11, 12, 13, 14, 15, 16, 17, 18, 19, or 20 amino acid segments (of two or more amino acids) or non-contiguous single amino acids. Additions (addition variants) include fusion proteins containing: (a) any of the enzymes described herein or a fragment thereof; and (b) internal or terminal (C or N) irrelevant or heterologous amino acid sequences. In the context of such fusion proteins, the term “heterologous amino acid sequences” refers to an amino acid sequence other than (a). A heterologous sequence can be, for example a sequence used for purification of the recombinant protein (e.g., FLAG, polyhistidine (e.g., hexahistidine), hemagluttanin (HA), glutathione-S-transferase (GST), or maltose binding protein (MBP)). Heterologous sequences also can be proteins useful as detectable markers, for example, luciferase, green fluorescent protein (GFP), or chloramphenicol acetyl transferase (CAT). In some embodiments, the fusion protein contains a signal sequence from another protein. In certain host cells (e.g., yeast host cells), expression and/or secretion of the target protein can be increased through use of a heterologous signal sequence. In some embodiments, the fusion protein can contain a carrier (e.g., KLH) useful, e.g., in eliciting an immune response for antibody generation) or ER or Golgi apparatus retention signals. Heterologous sequences can be of varying length and in some cases can be a longer sequences than the full-length target proteins to which the heterologous sequences are attached.

Endogenous genes of the organisms altered for use in the present invention also can be disrupted to prevent the formation of undesirable metabolites or prevent the loss of intermediates in the pathway through other enzymes acting on such intermediates. In one nonlimiting embodiment, the organism used in the present invention is further altered to to interfere with one or more genes involved in the degradation of 3-HP. In one nonlimiting embodiment, one or more of the genes prpC1, mmsA1, mmsA2, mmsA3, hpdH, mmsB and/or one or more genes encoding a glycerol kinase, a CoA transferase or ligase and/or one or more enzymes converting 3-hydroxypropionate to succinyl-CoA are interfered with. In one nonlimiting embodiment, two or more of these genes are interfered with and/or more than one gene in a class of enzymes is interfered with.

In one nonlimiting embodiment, the organism is further modified to eliminate phaCAB, involved in PHBs production and/or H16-A0006-9 encoding endonucleases thereby improving transformation efficiency.

Thus, as described herein, altered organisms can include exogenous nucleic acids encoding a glycerol dehydratase and/or a glycerol dehydratase reactivase and/or an aldehyde dehydrogenase and/or a glycerol 3-phosphate phosphatase and/or a glycerol 3-phosphate dehydrogenase, as described herein, as well as modifications to endogenous genes.

The term “exogenous” as used herein with reference to a nucleic acid (or a protein) and an organism refers to a nucleic acid that does not occur in (and cannot be obtained from) a cell of that particular type as it is found in nature or a protein encoded by such a nucleic acid. Thus, a non-naturally-occurring nucleic acid is considered to be exogenous to a host once in the host. It is important to note that non-naturally-occurring nucleic acids can contain nucleic acid subsequences or fragments of nucleic acid sequences that are found in nature provided the nucleic acid as a whole does not exist in nature. For example, a nucleic acid molecule containing a genomic DNA sequence within an expression vector is non-naturally-occurring nucleic acid, and thus is exogenous to a host cell once introduced into the host, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid. A nucleic acid that is naturally-occurring can be exogenous to a particular host microorganism. For example, an entire chromosome isolated from a cell of yeast x is an exogenous nucleic acid with respect to a cell of yeast y once that chromosome is introduced into a cell of yeast y.

In contrast, the term “endogenous” as used herein with reference to a nucleic acid (e.g., a gene) (or a protein) and a host refers to a nucleic acid (or protein) that does occur in (and can be obtained from) that particular host as it is found in nature. Moreover, a cell “endogenously expressing” a nucleic acid (or protein) expresses that nucleic acid (or protein) as does a host of the same particular type as it is found in nature. Moreover, a host “endogenously producing” or that “endogenously produces” a nucleic acid, protein, or other compound produces that nucleic acid, protein, or compound as does a host of the same particular type as it is found in nature.

The present invention also provides exogenous genetic molecules of the nonnaturally occurring organisms disclosed herein such as, but not limited to, codon optimized nucleic acid sequences, expression constructs and/or synthetic operons.

In one nonlimiting embodiment, the exogenous genetic molecule comprises a codon optimized nucleic acid sequence encoding a glycerol dehydratase, a glycerol dehydratase reactivase, and/or an aldehyde dehydrogenase and/or a glycerol 3-phosphate phosphatase and/or a glycerol 3-phosphate dehydrogenase. In one nonlimiting embodiment, the nucleic acid sequence is codon optimized for C. necator.

In one nonlimiting embodiment, the exogenous genetic molecule comprises a nucleic acid sequence encoding Klebsiella pneumoniae glycerol dehydratase. In one nonlimiting embodiment, the exogenous genetic molecule comprises SEQ ID NO: 1 or 3 and/or 4 and/or 6, a nucleic acid sequence exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 1 or 3 and/or 4 and/or 6 or a functional fragment thereof, or a functional fragment thereof, or a nucleic acid sequence encoding a polypeptide with at least about 50%, 60%, 70%, 75%, 80%, 85%, 90%, 91%, 92%, 93%, 94%, 95%, 96%, 97%, 98%, 99% or 99.5% sequence identity to the polypeptide encoded by the nucleic acid sequence set forth in SEQ ID NO:1 or 3 and/or 4 and/or 6 and exhibiting similar enzymatic activities to this polypeptide.

In one nonlimiting embodiment, the exogenous genetic molecule comprises a nucleic acid encoding Klebsiella pneumoniae glycerol dehydratase reactivase. In one nonlimiting embodiment, the glycerol dehydratase reactivase comprises SEQ ID NO:9 and/or 10 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:9 and/or 10 or a functional fragment thereof. In one nonlimiting embodiment, the exogenous genetic molecule comprises SEQ ID NO: 8, a nucleic acid sequence exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 8 or a functional fragment thereof, or a nucleic acid sequence encoding a polypeptide with at least about 50%, 60%, 70%, 75%, 80%, 85%, 90%, 91%, 92%, 93%, 94%, 95%, 96%, 97%, 98%, 99% or 99.5% sequence identity to the polypeptide encoded by the nucleic acid sequence set forth in set forth in SEQ ID NO:8 or a functional fragment thereof and exhibiting similar enzymatic activities to this polypeptide.

In one nonlimiting embodiment, the exogenous genetic molecule comprises a nucleic acid encoding an aldehyde dehydrogenase from Klebsiella pneumoniae or E. coli. In one nonlimiting embodiment, the exogenous genetic molecule comprises SEQ ID NO: 11 or 13, a nucleic acid sequence exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 11 or 13 or a functional fragment thereof, or a nucleic acid sequence encoding a polypeptide with at least about 50%, 60%, 70%, 75%, 80%, 85%, 90%, 91%, 92%, 93%, 94%, 95%, 96%, 97%, 98%, 99% or 99.5% sequence identity to the polypeptide encoded by the nucleic acid sequence set forth in SEQ ID NO:11 or SEQ ID NO:13 or a functional fragment thereof and exhibiting similar enzymatic activities to this polypeptide.

In one nonlimiting embodiment, the exogenous genetic molecule comprises a nucleic acid sequence encoding a glycerol 3-phosphate phosphatase from S. cerevisiae. In one nonlimiting embodiment, the exogenous genetic molecule comprises a nucleic acid sequence encoding SEQ ID NO: 17, a nucleic acid sequence exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 17 or a functional fragment thereof, or a nucleic acid sequence encoding a polypeptide with similar enzymatic activities and at least about 50%, 60%, 70%, 75%, 80%, 85%, 90%, 91%, 92%, 93%, 94%, 95%, 96%, 97%, 98%, 99% or 99.5% sequence identity to the polypeptide encoded by SEQ ID NO:17 or a functional fragment thereof. In one nonlimiting embodiment, the exogenous genetic molecule comprises a nucleic acid sequence encoding a glycerol 3-phosphate dehydrogenase from S. cerevisiae. In one nonlimiting embodiment, the exogenous genetic molecule comprises a nucleic acid sequence encoding SEQ ID NO: 15, a nucleic acid sequence exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 15 or a functional fragment thereof, or a nucleic acid sequence encoding a polypeptide with similar enzymatic activities and at least about 50%, 60%, 70%, 75%, 80%, 85%, 90%, 91%, 92%, 93%, 94%, 95%, 96%, 97%, 98%, 99% or 99.5% sequence identity to the polypeptide encoded by SEQ ID NO:15 or a functional fragment thereof.

Additional nonlimiting examples of exogenous genetic molecules include expression constructs of, for example, a glycerol dehydratase and/or a glycerol dehydratase reactivase and/or an aldehyde dehydrogenase and/or a glycerol 3-phosphate phosphatase and/or a glycerol 3-phosphate dehydrogenase and synthetic operons of, for example a glycerol dehydratase and/or a glycerol dehydratase reactivase and/or an aldehyde dehydrogenase and/or a glycerol 3-phosphate phosphatase and/or a glycerol 3-phosphate dehydrogenase.

Expression of a glycerol dehydratase, dhaB, and a glycerol dehydratase reactivase, gdrAB, both of Klebsiella pneumoniae, and an aldehyde dehydrogenase puuC of K. pneumoniae or an aldehyde dehydrogenase aldH of E. coli classified in EC 1.2.1.B6 was carried out in C. necator to assess the carbon flux of the fructose node via 3-hydroxypropionic acid production.

H16 ΔphaCAB ΔA0006-9 was selected as a base strain for the analysis of 3-hydroxypropionate production in accordance with the methods and altered organisms of the present invention. Additional genes were selected to knock out in this strain that are expected to be involved in the degradation of 3-HP in C. necator, prpC1, mmsA1, mmsA2, mmsA3, hpdH and mmsB, resulting in strain H16 ΔphaCAB ΔA0006-9 ΔmmsA1 ΔprpC1 ΔmmsA2 ΔhpdH ΔmmsA3 ΔmmsB.

The prpC1 gene encodes a 2-methylcitrate synthase involved in the conversion of propanoyl-CoA into 2-methylcitrate. Its deletion in C. necator stops propanoate degradation via the methylcitrate cycle. Further, a propionate CoA-transferase with high specificity for 3-HP has been described in C. necator in in vitro experiments (Lindenkamp et al. Appl Microbiol Biotechnol 2013 97:7699-7709). Synthesis of 3-HP-CoA may lead to degradation of 3-HP through its conversion to acryloyl-CoA, then propanoyl-CoA, and finally entry into the methylcitrate cycle. While blocking the methylcitrate cycle would not stop completely the degradation of 3-HP, it could be diverted to propanoate synthesis. Deletion of this propionate CoA-transferase in C. necator did not show any phenotype (Lindenkamp et al. Appl Microbiol Biotechnol 2013 97:7699-7709); this may be due to the presence of other CoA transferases in this organism replacing its activity.

The mmsA2 gene encodes a methylmalonate-semialdehyde dehydrogenase enzyme involved in the conversion of malonate semialdehyde into acetyl-CoA. This enzyme has been shown to be upregulated in C. necator in the presence of 3-HP in the media, suggesting it could be involved in the catabolism of 3-HP in this organism. There are also two other copies of mmsA (mmsA1 and mmsA3) in C. necator.

Pseudomonas denitrificans can grow on 3-hydroxypropionic acid as a carbon source and can also degrade it in non-growing conditions. The enzymes involved in the catabolism of 3-HP to acetyl-CoA have been identified. The first step of the degradation is catalyzed by a 3-hydroxypropionate dehydrogenase (HpdH), and the second one, by a methylmalonate-semialdehyde dehydrogenase (MmsA). In vitro analysis also showed that a 3-hydroxyisobutyrate dehydrogenase (HbdH-4, also called MmsB) exhibits 3-hydroxypropionate degradation activity. In this organism, these genes are regulated by LysR-type transcriptional regulators (LTTR) which induce the expression of these genes in the presence of 3-HP (Zhou et al. Biotechnology for Biofuels 2015 8:169). Homologs of these genes have been described in C. necator, although the distribution is different from P. denitrificans and only one of the copies of mmsA, and hpdH, found in the same operon, are regulated by a LTTR. 3-HP inducible expression systems have been developed which are composed of a LysR-type transcriptional regulator and a 3-HP responsive promoter derived from P. denitrificans and C. necator (Hanko et al., Scientific Reports 2017 7, Article number: 1724).

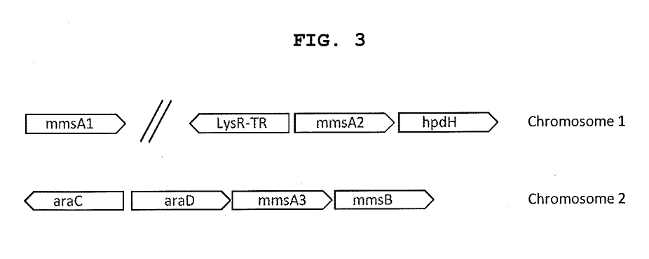

The distribution of these genes in the genome of C. necator is represented in FIG. 3.

Deletion of hpdH and mmsB in P. denitrificans led to the blockage of the degradation of this compound (Zhou et al. Appl Microbiol Biotechnol 2014 98:4389-4398). Therefore, deletion of these genes was carried out in C. necator ΔphaCAB ΔA0006-9, although all copies of mmsA were deleted as well. Specifically, three sequential deletions were done to delete mmsA1 (H16_RS01335), and the two operons containing the genes mmsA2 (H16_RS18295) and hpdH (H16_RS18290), and mmsA3 (H16_RS24710) and mmsB (H16_RS24705).

Two P_{BAD} promoters driven by only one araC regulatory gene were used.

The glycerol dehydratase reactivation factor, gdrAB was included due to the possibility of the glycerol dehydratase being inactivated by glycerol and/or oxygen and to allow for performance of the assay in aerobic conditions.

Additionally, the gene GPP2 from S. cerevisiae which encodes a glycerol 3-phosphate phosphatase was included in the expression vector as C. necator lacks this enzyme, necessary for the production of glycerol from glycerol 3-phosphate. The gene GPD1 from S. cerevisiae was also included.

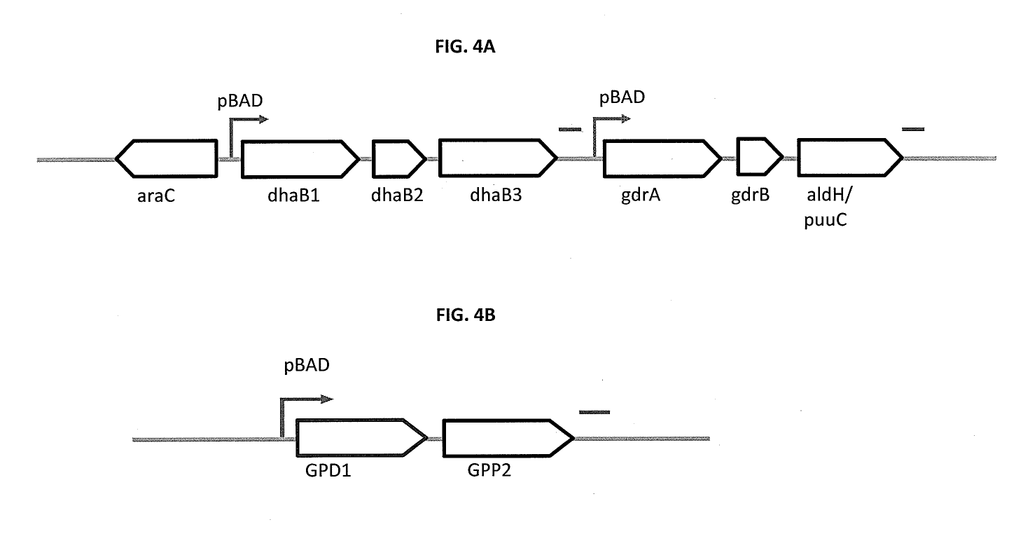

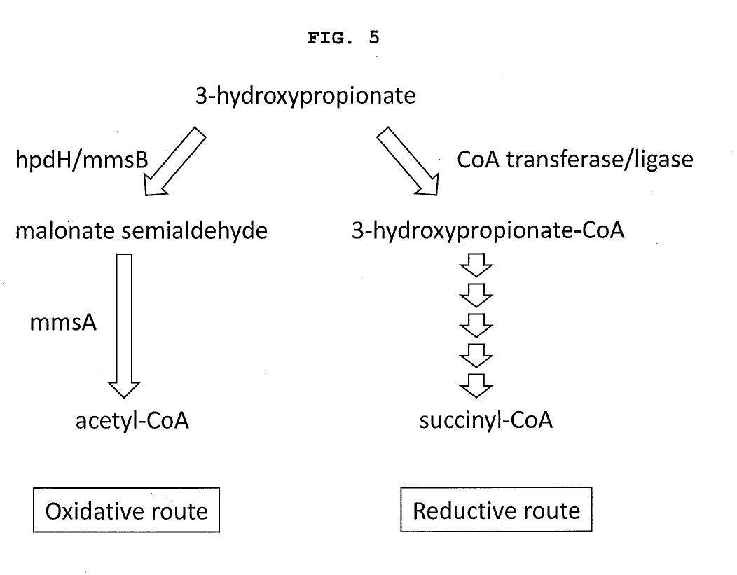

Distribution of these genes in pBBR1-1A and pMOL28-2A is represented in FIGS. 4A and 4B, respectively.

In E. coli, it has been shown that the intermediate 3-hydroxypropionaldehyde is toxic for the cell, impairing growth when this intermediate accumulates. In E. coli, modulation of the expression of the first gene, dhaB1, showed differences in cell growth and 3-HP production, being improved with the lowest expression of it (Raj et al. Appl Microbiol Biotechnol 2009 84:649). For this reason, a different version of each plasmid was constructed by replacing in dhaB1 the canonical RBS for C. necator with a ‘weak’ RBS, corresponding to RBS-E described by Zelcbuch et al. (Nucleic Acids Research 2013 41(9):e98).

C. necator H16 ΔphaCAB ΔA0006-9 and C. necator H16 ΔphaCAB ΔA0006-9 ΔmmsA1 ΔprpC1 ΔmmsA2 ΔhpdH ΔmmsA3 ΔmmsB were transformed with the resulting plasmids.

Also provided by the present invention are 3-HP and derivatives and compounds related thereto bioderived from an altered organism according to any of methods described herein.

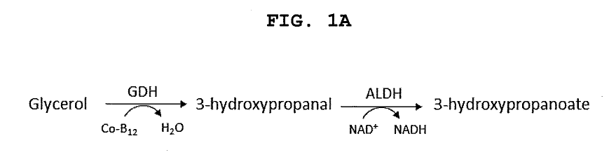

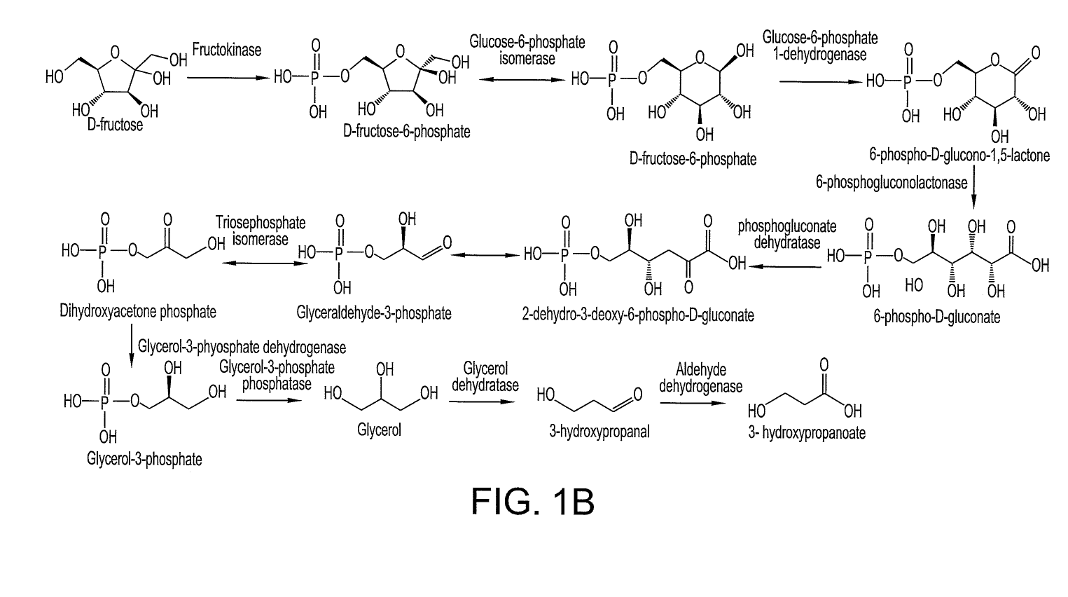

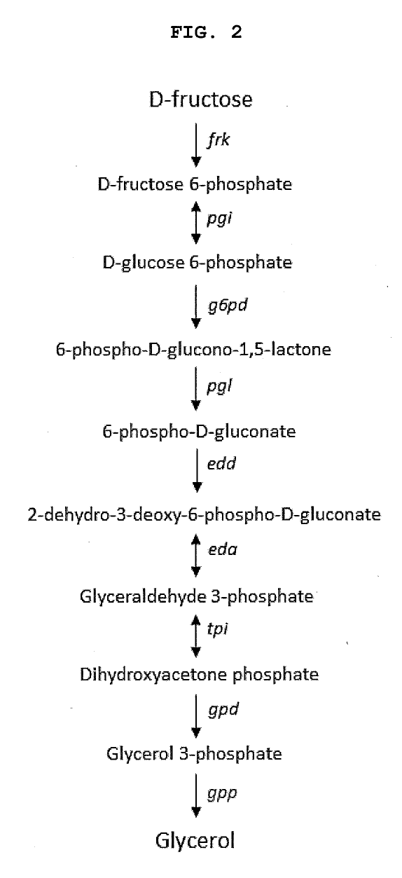

Further, the present invention relates to means and processes for use of these means for biosynthesis of 3-HP including derivatives thereof and/or compounds related thereto. Nonlimiting examples of such means include altered organisms and exogenous genetic molecules as described herein as well as any of the molecules as depicted in FIG. 1A, 1B, 2 or 5.

In addition, the present invention provides bio-derived, bio-based, or fermentation-derived products produced using the methods and/or altered organisms disclosed herein. In one nonlimiting embodiment, a bio-derived, bio-based or fermentation derived product is produced in accordance with the exemplary central metabolism depicted in FIG. 1B. Examples of such products include, but are not limited to, compositions comprising at least one bio-derived, bio-based, or fermentation-derived compound or any combination thereof, as well as polymers, plastics, molded substances, formulations and semi-solid or non-semi-solid streams comprising one or more of the bio-derived, bio-based, or fermentation-derived compounds or compositions, combinations or products thereof.

The following section provides further illustration of the methods and materials of the present invention. These Examples are illustrative only and are not intended to limit the scope of the invention in any way.

### EXAMPLES

### Strains and Plasmids

E. coli DH5a (New England Biolabs) was used as a host for plasmid construction.

H16 ΔphaCAB ΔA0006-9 and H16 ΔphaCAB ΔA0006-9 ΔmmsA1 ΔprpC1 ΔmmsA2 ΔhpdH ΔmmsA3 ΔmmsB were used as base C. necator strains for the expression of the 3-hydroxypropionic acid pathway.

Sequences for C. necator of the genes specified in Table 1 were synthesized:

TABLE 1
List of genes expressed

| GenBank # | Gene |
|---|---|
| AAA74258.1 | Klebsiella pneumoniae |
| | dhaB1 |
| AAA74256.1 | Klebsiella pneumoniae |
| | dhaB2 |
| AAA74255.1 | Klebsiella pneumoniae |
| | dhaB3 |
| NP_415816.1 | E. coli aldH |
| ABR76453.1 | Klebsiella pneumoniae |
| | puuC |
| ABO37963.1 | Klebsiella pneumoniae |
| | gdrA |
| ABO37964.1 | Klebsiella pneumoniae |
| | gdrB |
| NP_010984.3 | S. cerevisiae GPP2 |
| NP_010262.1 | S. cerevisiae GPD1 |

All plasmids were constructed using standard cloning techniques such as described, for example in Green and Sambrook, Molecular Cloning, A Laboratory Manual, Nov. 18, 2014. All constructs were verified by analytical PCR and then by sequencing as provided by eurofinsgenomics with the extension .eu/en/eurofins-genomics/product-faqs/custom-dna-sequencing/ of the world wide web.

Transformation of C. necator H16 ΔphaCAB ΔA0006-9 and H16 ΔphaCAB ΔA0006-9 ΔmmsA1 ΔprpC1 ΔmmsA2 ΔhpdH ΔmmsA3 ΔmmsB was performed following a standard electroporation technique. Strains obtained are listed in Table 2.

TABLE 2
List of strains used in this study

| Organism | Plasmid |
|---|---|
| E. coli DH5α | pBBR1-1A-araC-P_{BAD}-Sc_GPP2-Kp_dhaB123- |
| | rnpBT1-P_{BAD}-Kp_gdrAB-Ec_aldH-rrnBT2 |
| E. coli DH5α | pBBR1-1A-araC-P_{BAD}-Sc_GPP2-Kp_dhaB123- |
| | rnpBT1-P_{BAD}-Kp_gdrAB-Kp_puuC-rrnBT2 |
| E. coli DH5α | pBBR1-1A-araC-P_{BAD}-Sc_GPP2-RBS_E-Kp_dhaB1- |
| | Kp_dhaB23rnpBT1-P_{BAD}-Kp_gdrAB-Ec_aldH-rrnBT2 |
| E. coli DH5α | pBBR1-1A-araC-P_{BAD}-Sc_GPP2-RBS_E-Kp_dhaB1- |
| | Kp_dhaB23-rnpBT1-P_{BAD}-Kp_gdrAB-Kp_puuC-rrnBT2 |
| C. necator H16 ΔphaCAB | pBBR1-1A-araC-P_{BAD} |
| ΔA0006-9 | |
| C. necator H16 ΔphaCAB | pBBR1-1A-araC-P_{BAD}-Kp_dhaB123-rnpBT1-P_{BAD}- |
| ΔA0006-9 | Kp_gdrAB-Ec_aldH-rrnBT2, pMOL28-2A-P_{BAD}- |
| | Sc_GPD1-Sc_GPP2-rrnBt1T2 |
| C. necator H16 ΔphaCAB | pBBR1-1A-araC-P_{BAD}-Kp_dhaB123-rnpBT1-P_{BAD}- |
| ΔA0006-9 | Kp_gdrAB-Kp_puuC-rrnBT2, pMOL28-2A-P_{BAD}- |
| | Sc_GPD1-Sc_GPP2-rrnBt1T2 |
| C. necator H16 ΔphaCAB | pBBR1-1A-araC-P_{BAD}-RBS-E-Kp_dhaB1-Kp_dhaB23- |
| ΔA0006-9 | rnpBT1-P_{BAD}-Kp_gdrAB-Ec_aldH-rrnBT2, pMOL28- |
| | 2A-P_{BAD}-Sc_GPD1-Sc_GPP2-rrnBt1T2 |
| C. necator H16 ΔphaCAB | pBBR1-1A-araC-P_{BAD}-RBS_E-Kp_dhaB1-Kp_dhaB23- |
| ΔA0006-9 | rnpBT1-P_{BAD}-Kp_gdrAB-Kp_puuC-rrnBT2, pMOL28- |
| | 2A-P_{BAD}-Sc_GPD1-Sc_GPP2-rrnBt1T2 |
| C. necator H16 ΔphaCAB | pBBR1-1A-araC-P_{BAD}, pMOL28-2A |
| ΔA0006-9 ΔmmsA1 | |
| ΔprpC1 ΔmmsA2 | |
| ΔhpdH ΔmmsA3 ΔmmsB | |
| C. necator H16 ΔphaCAB | pBBR1-1A-araC-P_{BAD}-Kp_dhaB123-rnpBT1-P_{BAD}- |
| ΔA0006-9 ΔmmsA1 | Kp_gdrAB-Ec_aldH-rrnBT2, pMOL28-2A-P_{BAD}- |
| ΔprpC1 ΔmmsA2 | Sc_GPD1-Sc_GPP2-rrnBt1T2 |
| ΔhpdH ΔmmsA3 ΔmmsB | |
| C. necator H16 ΔphaCAB | pBBR1-1A-araC-P_{BAD}-Kp_dhaB123-rnpBT1-P_{BAD}- |
| ΔA0006-9 ΔmmsA1 | Kp_gdrAB-Kp_puuC-rrnBT2, pMOL28-2A-P_{BAD}- |
| ΔprpC1 ΔmmsA2 | Sc_GPD1-Sc_GPP2-rrnBt1T2 |
| ΔhpdH ΔmmsA3 ΔmmsB | |
| C. necator H16 ΔphaCAB | pBBR1-1A-araC-P_{BAD}-RBS_E-Kp_dhaB1-Kp_dhaB23- |
| ΔA0006-9 ΔmmsA1 | rnpBT1-P_{BAD}-Kp_gdrAB-Ec_aldH-rrnBT2, pMOL28- |
| ΔprpC1 ΔmmsA2 | 2A-P_{BAD}-Sc_GPD1-Sc_GPP2-rrnBt1T2 |
| ΔhpdH ΔmmsA3 ΔmmsB | |
| C. necator H16 ΔphaCAB | pBBR1-1A-araC-P_{BAD}-RBS_E-Kp_dhaB1-Kp_dhaB23- |
| ΔA0006-9 ΔmmsA1 | rnpBT1-P_{BAD}-Kp_gdrAB-Kp_puuC-rrnBT2, pMOL28- |
| ΔprpC1 ΔmmsA2 | 2A-P_{BAD}-Sc_GPD1-Sc_GPP2-rrnBt1T2 |
| ΔhpdH ΔmmsA3 ΔmmsB | |

Sc: Saccharomyces cerevisiae; Kp: Klebsiella pneumoniae; Ec: Escherichia coli.

LB media was used to grow and maintain E. coli strains. Appropriate antibiotic was added when required. TSB was used to grow and maintain C. necator strains. Appropriate antibiotic was added when required. A minimal medium as shown in Table 3 was used to grow C. necator strains for 3-HP production.

TABLE 3

| | |
|---|---|
| Component | g/L |

Base composition

| | |
|---|---|
| Fructose | 12 |
| Nitrilotriacetic acid | 0.15 |
| KH₂PO₄ | 1.4 |
| Na₂HPO₄ | 0.94 |
| (NH₄)₂SO₄ | 3.365 |
| MgSO₄•7H₂O | 0.5 |
| CaCl₂•2H₂O | 0.01 |
| NH₄Fe(II)SO₄•6H₂O | 0.05 |
| Trace metal solution | 10 ml |

Trace metal solution composition

| | |
|---|---|
| ZnSO₄•7H₂O | 0.1 |
| MnCl₂•4H₂O | 0.03 |
| H₃BO₃ | 0.3 |
| CoCl₂•6H₂O | 0.2 |
| NiSO₄•6H₂O | 0.025 |
| Na₂MoO₄•2H₂O | 0.03 |
| CuSO₄•5H₂O | 0.015 |

Examples of hpdH and mmsB Enzymes which May be Altered

Nonlimiting examples of 3-hydroxyisobutyrate dehydrogenase, 2-hydroxy-3-oxopropionate reductase and NAD-dependent beta-hydroxyacid dehydrogenase referred to collectively as mmsB, and choline dehydrogenase, glucose-methanol-choline oxidoreductase and oxidoreductase referred to collectively as hpdH, which convert 3-hydroxypropionate to malonate semialdehyde are disclosed in Table 4. Experiments have been conducted where H16_A3663 and/or H16-B1190 of C. necator have been deleted. However, as will be understood by the skilled artisan upon reading this disclosure, more than one of these polypeptides and enzymes may be altered for use in accordance with the present invention.

TABLE 4

% Identity (>30%), covering
>90% of sequence*

| | Enzyme/ | C. necator | P. denitrificans |
|---|---|---|---|
| hpdH | Accession No. | H16_A3663 | YP_007659112 |
| H16_A3663 | choline | 100 | 60 |
| | dehydrogenase | | |
| | WP_010811289.1 | | |
| H16_B1851 | glucose-methanol- | 46 | 46 |
| | choline | | |
| | oxidoreductase | | |
| | WP_010810328.1 | | |
| H16_B1532 | Oxidoreductase | 43 | 42 |
| | WP_011617294.1 | | |
| H16_B2131 | choline | 41 | 41 |
| | dehydrogenase | | |
| | WP_010811005.1 | | |
| H16_A0233 | choline | 39 | 41 |
| | dehydrogenase | | |
| | WP_011614415.1 | | |
| H16_B0411 | choline | 39 | 41 |
| | dehydrogenase and | | |
| | alkyl sulfatase | | |
| | WP_011616571.1 | | |

| | | % Identity (>30%), covering >90% of sequence | | |
|---|---|---|---|---|
| | | C. necator | P. denitrificans | P. denitrificans |
| mmsB | Enzyme/Accession No. | H16_B1190 | YP_007656737 | YP_007658098 |
| H16_B1190 | 3-hydroxyisobutyrate | 100 | 52 | 66 |
| | dehydrogenase | | | |
| | WP_011617070.1 | | | |
| H16_B1750 | 3-hydroxyisobutyrate | 45 | 44 | 46 |
| | dehydrogenase | | | |
| | WP_011617453.1 | | | |
| H16_A3004 | 3-hydroxyisobutyrate | 38 | 37 | 38 |
| | dehydrogenase | | | |
| | WP_010814951.1 | | | |
| H16_B1657 | 3-hydroxyisobutyrate | 34 | 33 | |
| | dehydrogenase | | | |
| | WP_011617380.1 | | | |
| H16_A3600 | 2-hydroxy-3- | 35 | 33 | 35 |
| | oxopropionate | | | |
| | reductase | | | |
| | WP_010812149.1 | | | |
| H16_B0941 | NAD-dependent beta- | 36 | 35 | 39 |
| | hydroxyacid | | | |
| | dehydrogenase | | | |
| | WP_010809660.1 | | | |
| H16_A1562 | 3-hydroxyisobutyrate | 31 | 31 | 37 |
| | dehydrogenase | | | |
| | WP_011615152.1 | | | |
| H16_A1239 | 3-hydroxyisobutyrate | 30 | | |
| | dehydrogenase | | | |
| | WP_011614949.1 | | | |

*by % Identity (>30%), covering >90% of sequence it is meant that the genes all have at least 30% sequence identity along at least any 90% of the length, relative to the first C. necator gene listed which has already been knocked out.

Examples of CoA Transferase or Ligase Enzymes which May be Altered

Nonlimiting examples of CoA transferase or ligase enzymes which convert 3-hydroxypropionate to 3-hydroxypropionate-CoA are disclosed in SEQ ID NOs: 19 through 34. See Fukui et al. Biomacromolecules 2009 13 10(4):700-6 and Volodina et al. Appl Microbiol Biotechnol. 2014 98(8): 3579-89. As will be understood by the skilled artisan upon reading this disclosure, more than one polypeptide or enzyme may be altered for use in accordance with the present invention.

### Bioassay for 3-HP Analysis

Pre-cultures were prepared using standard procedures. Cells were subsequently washed in a defined minimal media (see Table 3) before inoculation. After growth upon the defined minimal media, cells were induced with L-Arabinose. 18 h and/or 24 h after induction, samples were taken by centrifuging the culture and collecting 1 ml supernatant. Pellets were frozen for the analysis of possible 3-HP polymers.

### LC-MS Analysis of 3-HP

Analysis of 3-hydroxypropionate was performed by LC-MS.

GC-MS Analysis of by-Products

Analysis of all by-products was performed by GC-MS.

### Sequence Information for Sequences in Sequence Listing

TABLE 5

| SEQ | |
|---|---|
| ID | |
| NO: | Sequence Description |
| 1 | Nucleic acid sequence of AAA74258.1 (dhaB1) |
| 2 | Amino acid sequence of AAA74258.1 (dhaB1) |
| 3 | Nucleic acid sequence of Weak RBS-AAA74258.1 (dhaB1) |
| 4 | Nucleic acid sequence of AAA74256.1 (dhaB2) |
| 5 | Amino acid sequence OF AAA74256.1 (dhaB2) |
| 6 | Nucleic acid sequence of AAA74255.1 (dhaB3) |
| 7 | Amino acid sequence of AAA74255.1 (dhaB3) |
| 8 | Nucleic acid sequence of ABO37963.1-ABO37964.1 (gdrA,B) |
| 9 | Amino acid sequence of ABO37963.1 |
| 10 | Amino acid sequence of ABO37964.1 |
| 11 | Nucleic acid sequence of NP_415816.1 (E. coli aldH) |
| 12 | Amino acid sequence of NP_415816.1 (E. coli aldH) |
| 13 | Nucleic acid sequence of ABR76453.1 (Klebsiella |
| | pneumoniae puuC) |
| 14 | Amino acid sequence of ABR76453.1 (Klebsiella pneumoniae |
| | puuC) |
| 15 | Nucleic acid sequence of NP_010262.1 (S. cerevisiae GPD1) |
| 16 | Amino acid sequence of NP_010262.1 (S. cerevisiae GPD1) |
| 17 | Nucleic acid sequence of NP_010984.3 (S. cerevisiae GPP2) |
| 18 | Amino acid sequence of NP_010984.3 (S. cerevisiae GPP2) |
| 19 | Amino acid sequence of PROPIONATE COA-TRANSFERASE |
| | (PCT); EC 2.8.3.1; H16_A2718; CAJ93797 |
| 20 | Nucleic acid sequence of PROPIONATE COA-TRANSFERASE |
| | (PCT); EC 2.8.3.1; H16_A2718; CAJ93797 |
| 21 | Amino acid sequence of PROPIONATE-COA LIGASE (PRPE); |
| | EC 6.2.1.17 H16_A2462; CAJ93551 |
| 22 | Nucleic acid sequence of PROPIONATE-COA LIGASE (PRPE); |
| | EC 6.2.1.17 H16_A2462; CAJ93551 |
| 23 | Amino acid sequence of ACETYL-COA SYNTHETASE/LIGASE; |
| | EC 6.2.1.1 H16_A1197; CAJ92338 |
| 24 | Nucleic acid sequence of ACETYL-COA SYNTHETASE/ |
| | LIGASE; EC 6.2.1.1 H16_A1197; CAJ92338 |
| 25 | Amino acid sequence of EC 6.2.1.1 H16_A1616; CAJ92748 |
| 26 | Nucleic acid sequence of EC 6.2.1.1 H16_A1616; CAJ92748 |
| 27 | Amino acid sequence of EC 6.2.1.1 H16_A2525; CAJ93612 |
| 28 | Nucleic acid sequence of EC 6.2.1.1 H16_A2525; CAJ93612 |
| 29 | Amino acid sequence of EC 6.2.1.1 H16_B0396; CAJ95185 |
| 30 | Nucleic acid sequence of EC 6.2.1.1 H16_B0396; CAJ95185 |
| 31 | Amino acid sequence of EC 6.2.1.1 H16_B0834; CAJ95626 |
| 32 | Nucleic acid sequence of EC 6.2.1.1 H16_B0834; CAJ95626 |
| 33 | Amino acid sequence of EC 6.2.1.1 H16_B1102; CAJ95893 |
| 34 | Nucleic acid sequence of EC 6.2.1.1 H16_B1102; CAJ95893 |

1: A process for biosynthesis of 3-hydroxypropanoic acid (3-HP), derivatives thereof and/or compounds related thereto, said process comprising:
obtaining an organism capable of producing 3-HP, derivatives thereof and/or compounds related thereto;
altering the organism; and
producing more 3-HP, derivatives thereof and/or compounds related thereto by the altered organism as compared to the unaltered organism.

2: The process of claim 1 wherein the organism is C. necator or an organism with properties similar thereto.

3: The process of claim 1 wherein the organism is altered to express one or more of a glycerol dehydratase and/or a glycerol dehydratase reactivase and/or an aldehyde dehydrogenase and/or a glycerol 3-phosphate phosphatase and/or glycerol 3-phosphate dehydrogenase.

4-7. (canceled)

8: The process of claim 3 wherein the glycerol dehydratase is from Klebsiella pneumoniae, the glycerol dehydratase reactivase is from Klebsiella pneumoniae, the aldehyde dehydrogenase is from Klebsiella pneumoniae or E. coli, the glycerol 3-phosphate phosphatase is GPP2 from S. cerevisiae and/or the glycerol 3-phosphate dehydrogenaseis GPD1 from S. cerevisiae.

9: The process of claim 3 wherein the glycerol dehydratase comprises:
SEQ ID NO:2, 5 and/or 7;
a polypeptide with similar enzymatic activities exhibiting at least about 50% sequence identity to an amino acid sequence set forth in SEQ ID NO:2, 5 and/or 7 or a functional fragment thereof;
a polypeptide encoded by a nucleic acid sequence of SEQ ID NO: 1 or 3 and/or 4 and/or 6;
a polypeptide with similar enzymatic activities exhibiting at least about 50% sequence identity to a polypeptide encoded by a nucleic acid sequence set forth in SEQ ID NO: 1 or 3 and/or 4 and/or 6 or a functional fragment thereof; or
a polypeptide with similar enzymatic activities encoded by a nucleic acid sequence with at least about 50% sequence identity to the nucleic acid sequence set forth in SEQ ID NO: 1 or 3 and/or 4 and/or 6 or a functional fragment thereof.

10. (canceled)

11: The process of claim 3 wherein the glycerol dehydratase reactivase comprises:
SEQ ID NO:9 and/or 10;
a polypeptide with similar enzymatic activities exhibiting at least about 50% sequence identity to an amino acid sequence set forth in SEQ ID NO:9 and/or 10 or a functional fragment thereof;
a polypeptide encoded by a nucleic acid sequence of SEQ ID NO: 8;
a polypeptide with similar enzymatic activities exhibiting at least about 50% sequence identity to a polypeptide encoded by the nucleic acid sequence set forth in SEQ ID NO: 8 or a functional fragment thereof; or
a polypeptides with similar enzymatic activities encoded by a nucleic acid sequence with at least 50% sequence identity to the nucleic acid sequence set forth in SEQ ID NO:8 or a functional fragment thereof.

12. (canceled)

13: The process of claim 3 wherein the aldehyde dehydrogenase comprises:
SEQ ID NO:12 or 14;
a polypeptide with similar enzymatic activities exhibiting at least about 50% sequence identity to an amino acid sequence set forth in SEQ ID NO:12 or 14 or a functional fragment thereof;
a polypeptide encoded by a nucleic acid sequence of SEQ ID NO: 11 or 13;
a polypeptide with similar enzymatic activities exhibiting at least about 50% sequence identity to a polypeptide encoded by the nucleic acid sequence set forth in SEQ ID NO:11 or 13 or a functional fragment thereof; or
a polypeptide with similar enzymatic activities encoded by a nucleic acid sequence with at least about 50% sequence identity to the nucleic acid sequence set forth in SEQ ID NO:11 or SEQ ID NO:13 or a functional fragment thereof.

14. (canceled)

15: The process of claim 3 wherein the glycerol 3-phosphate phosphatase comprises:
SEQ ID NO:18;
a polypeptide with similar enzymatic activities exhibiting at least about 50% sequence identity to an amino acid sequence set forth in SEQ ID NO:18 or a functional fragment thereof;
a polypeptide encoded by a nucleic acid sequence of SEQ ID NO: 17;
a polypeptide with similar enzymatic activities exhibiting at least about 50% sequence identity to a polypeptide encoded by the nucleic acid sequence set forth in SEQ ID NO:17 or a functional fragment thereof; or a polypeptide with similar enzymatic activities encoded by a nucleic acid sequence with at least about 50% sequence identity to the nucleic acid sequence set forth in SEQ ID NO:17 or a functional fragment thereof.

16. (canceled)

17: The process of claim 3 wherein the glycerol 3-phosphate dehydrogenase comprises:
SEQ ID NO:16;
a polypeptide with similar enzymatic activities exhibiting at least about 50% sequence identity to an amino acid sequence set forth in SEQ ID NO:16 or a functional fragment thereof;
a polypeptide encoded by a nucleic acid sequence of SEQ ID NO: 15;
a polypeptide with similar enzymatic activities exhibiting at least about 50% sequence identity to a polypeptide encoded by the nucleic acid sequence set forth in SEQ ID NO: 15 or a functional fragment thereof; or
a polypeptide with similar enzymatic activities encoded by a nucleic acid sequence with at least about 50% sequence identity to the nucleic acid sequence set forth in SEQ ID NO:15 or a functional fragment thereof.

18: The process of claim 1 wherein the organism is further altered to interfere with one or more genes involved in the degradation of 3-HP.

19: The process of claim 18 wherein the one or more genes is prpC1, mmsA1, mmsA2, mmsA3, hpdH, or mmsB or encodes a glycerol kinase, a CoA transferase or ligase or an enzyme converting 3-hydroxypropionate to succinyl-CoA.

20-31. (canceled)

32: The process of claim 1 wherein the organism is further altered to eliminate phaCAB, involved in PHBs production and/or H16-A0006-9 encoding endonucleases thereby improving transformation efficiency.

33. (canceled)

34: An altered organism capable of producing more 3-HP, derivatives thereof and/or compounds related thereto as compared to an unaltered organism.

35: The altered organism of claim 34 which is C. necator or an organism with properties similar thereto.

36: The altered organism of claim 34 which expresses one or more of a glycerol dehydratase and/or a glycerol dehydratase reactivase and/or an aldehyde dehydrogenase and/or a glycerol 3-phosphate phosphatase and/or a glycerol 3-phosphate dehydrogenase.

37-40. (canceled)

41: The altered organism of claim 36 wherein the glycerol dehydratase is from Klebsiella pneumoniae, the glycerol dehydratase reactivase is from Klebsiella pneumoniae, the aldehyde dehydrogenase is from Klebsiella pneumoniae or E. coli, the glycerol 3-phosphate phosphatase is GPP2 from S. cerevisiae and/or the glycerol 3-phosphate dehydrogenaseis GPD1 from S. cerevisiae.

42: The altered organism of claim 36 wherein the glycerol dehydratase comprises:
SEQ ID NO:2, 5 and/or 7;
a polypeptide with similar enzymatic activities exhibiting at least about 50% sequence identity to an amino acid sequence set forth in SEQ ID NO:2, 5 and/or 7 or a functional fragment thereof;
a polypeptide encoded by a nucleic acid sequence of SEQ ID NO: 1 or 3 and/or 4 and/or 6;
a polypeptide with similar enzymatic activities exhibiting at least about 50% sequence identity to a polypeptide encoded by a nucleic acid sequence set forth in SEQ ID NO: 1 or 3 and/or 4 and/or 6 or a functional fragment thereof; or
a polypeptide with similar enzymatic activities encoded by a nucleic acid sequence with at least about 50% sequence identity to the nucleic acid sequence set forth in SEQ ID NO: 1 or 3 and/or 4 and/or 6 or a functional fragment thereof.

43. (canceled)

44: The altered organism of claim 36 wherein the glycerol dehydratase reactivase comprises:
SEQ ID NO:9 and/or 10;
a polypeptide with similar enzymatic activities exhibiting at least about 50% sequence identity to an amino acid sequence set forth in SEQ ID NO:9 and/or 10 or a functional fragment thereof;
a polypeptide encoded by a nucleic acid sequence of SEQ ID NO: 8;
a polypeptide with similar enzymatic activities exhibiting at least about 50% sequence identity to a polypeptide encoded by the nucleic acid sequence set forth in SEQ ID NO: 8 or a functional fragment thereof; or
a polypeptides with similar enzymatic activities encoded by a nucleic acid sequence with at least about 50% sequence identity to the nucleic acid sequence set forth in SEQ ID NO:8 or a functional fragment thereof.

45. (canceled)

46: The altered organism of claim 36 wherein the aldehyde dehydrogenase comprises:
SEQ ID NO:12 or 14;
a polypeptide with similar enzymatic activities exhibiting at least about 50% sequence identity to an amino acid sequence set forth in SEQ ID NO:12 or 14 or a functional fragment thereof;
a polypeptide encoded by a nucleic acid sequence of SEQ ID NO: 11 or 13;
a polypeptide with similar enzymatic activities exhibiting at least about 50% sequence identity to a polypeptide encoded by the nucleic acid sequence set forth in SEQ ID NO: 11 or 13 or a functional fragment thereof; or
a polypeptide with similar enzymatic activities encoded by a nucleic acid sequence with at least about 50% sequence identity to the nucleic acid sequence set forth in SEQ ID NO:11 or SEQ ID NO:13 or a functional fragment thereof.

47. (canceled)

48: The altered organism of claim 36 wherein the glycerol 3-phosphate phosphatase comprises:
SEQ ID NO:18;
a polypeptide with similar enzymatic activities exhibiting at least about 50% sequence identity to an amino acid sequence set forth in SEQ ID NO:18 or a functional fragment thereof;
a polypeptide encoded by a nucleic acid sequence of SEQ ID NO: 17;
a polypeptide with similar enzymatic activities exhibiting at least about 50% sequence identity to a polypeptide encoded by the nucleic acid sequence set forth in SEQ ID NO:17 or a functional fragment thereof; or
a polypeptide with similar enzymatic activities encoded by a nucleic acid sequence with at least about 50% sequence identity to the nucleic acid sequence set forth in SEQ ID NO:17 or a functional fragment thereof.

49. (canceled)

50: The altered organism of claim 36 wherein the glycerol 3-phosphate dehydrogenase comprises:
SEQ ID NO:16;
a polypeptide with similar enzymatic activities exhibiting at least about 50% sequence identity to an amino acid sequence set forth in SEQ ID NO:16 or a functional fragment thereof;
a polypeptide encoded by a nucleic acid sequence of SEQ ID NO: 15;
a polypeptide with similar enzymatic activities exhibiting at least about 50% sequence identity to a polypeptide encoded by the nucleic acid sequence set forth in SEQ ID NO: 15 or a functional fragment thereof; or
a polypeptide with similar enzymatic activities encoded by a nucleic acid sequence with at least about 50% sequence identity to the nucleic acid sequence set forth in SEQ ID NO:15 or a functional fragment thereof.

51: The altered organism of claim 34 wherein the organism is further altered to interfered with one or more genes involved in the degradation of 3-HP.

52: The altered organism of claim 51 wherein the one or more genes is prpC1, mmsA1, mmsA2, mmsA3, hpdH, mmsB or encodes a glycerol kinase, a CoA transferase or ligase and/or an enzyme converting 3-hydroxypropionate to succinyl-CoA.

53-64. (canceled)

65: The altered organism of claim 34 wherein the organism is further altered to eliminate phaCAB, involved in PHBs production and/or H16-A0006-9 encoding endonucleases thereby improving transformation efficiency.

66. (canceled)

67: A bio-derived, bio-based, or fermentation-derived product produced from the method of claim 1, wherein said product comprises:
(i) a composition comprising at least one bio-derived, bio-based, or fermentation-derived compound or any combination thereof;
(ii) a bio-derived, bio-based, or fermentation-derived polymer comprising the bio-derived, bio-based, or fermentation-derived composition or compound of (i), or any combination thereof;
(iii) a bio-derived, bio-based, or fermentation-derived plastic comprising the bio-derived, bio-based, or fermentation-derived compound or bio-derived, bio-based, or fermentation-derived composition of (i), or any combination thereof or the bio-derived, bio-based, or fermentation-derived polymer of (ii), or any combination thereof;
(iv) a molded substance obtained by molding the bio-derived, bio-based, or fermentation-derived polymer of (ii), or the bio-derived, bio-based, or fermentation-derived plastic of (iii), or any combination thereof;
(v) a bio-derived, bio-based, or fermentation-derived formulation comprising the bio-derived, bio-based, or fermentation-derived composition of (i), the bio-derived, bio-based, or fermentation-derived compound of (i), the bio-derived, bio-based, or fermentation-derived polymer of (ii), the bio-derived, bio-based, or fermentation-derived plastic of (iii), or the bio-derived, bio-based, or fermentation-derived molded substance of (iv), or any combination thereof; or
(vi) a bio-derived, bio-based, or fermentation-derived semi-solid or a non-semi-solid stream, comprising the bio-derived, bio-based, or fermentation-derived composition of (i), the bio-derived, bio-based, or fermentation-derived compound of (i), the bio-derived, bio-based, or fermentation-derived polymer of (ii), the bio-derived, bio-based, or fermentation-derived plastic of (iii), the bio-derived, bio-based, or fermentation-derived formulation of (iv), or the bio-derived, bio-based, or fermentation-derived molded substance of (v), or any combination thereof.

68: A bio-derived, bio-based or fermentation derived product produced in accordance with the central metabolism depicted in FIG. 1B.

69: An exogenous genetic molecule of the altered organism of claim 34.

70: The exogenous genetic molecule of claim 69 comprising a codon optimized nucleic acid sequence or an expression construct or synthetic operon of a glycerol dehydratase and/or a glycerol dehydratase reactivase and/or an aldehyde dehydrogenase and/or a glycerol 3-phosphate phosphatase and/or a glycerol 3-phosphate dehydrogenase.

71: The exogenous genetic molecule of claim 70 codon optimized for C. necator.

72: The exogenous genetic molecule of claim 69 wherein the exogenous genetic molecule comprises a nucleic acid encoding Klebsiella pneumoniae glycerol dehydratase, Klebsiella pneumoniae glycerol dehydratase reactivase, an aldehyde dehydrogenase from Klebsiella pneumoniae or E. coli, a glycerol 3-phosphate phosphatase from S. cerevisiae or a glycerol 3-phosphate dehydrogenase from S. cerevisiae.

73: The exogenous genetic molecule of claim 69 comprising:
SEQ ID NO: 1 or 3 and/or 4 and/or 6;
a nucleic acid sequence with at least about 50% sequence identity to the nucleic acid sequence set forth in SEQ ID NO: 1 or 3 and/or 4 and/or 6 or a functional fragment thereof; or
a nucleic acid sequence encoding a polypeptide with similar enzymatic activities and exhibiting at least about 50% sequence identity to a polypeptide encoded by the nucleic acid sequence set forth in SEQ ID NO: 1 or 3 and/or 4 and/or 6 or a functional fragment thereof.

74. (canceled)

75: The exogenous genetic molecule of claim 69 comprising:
SEQ ID NO: 8;
a nucleic acid sequence with at least about 50% sequence identity to the nucleic acid sequence set forth in SEQ ID NO:8 or a functional fragment thereof; or
a nucleic acid sequence encoding a polypeptide with similar enzymatic activities and exhibiting at least about 50% sequence identity to a polypeptide encoded by the nucleic acid sequence set forth in SEQ ID NO:8 or a functional fragment thereof.

76. (canceled)

77: The exogenous genetic molecule of claim 69 comprising:
SEQ ID NO: 11 or 13;
a nucleic acid sequence with at least about 50% sequence identity to the nucleic acid sequence set forth in SEQ ID NO:11 or 13 or a functional fragment thereof; or
a nucleic acid sequence encoding a polypeptide with similar enzymatic activities and exhibiting at least about 50% sequence identity to a polypeptide encoded by the nucleic acid sequence set forth in SEQ ID NO:11 or 13 or a functional fragment thereof.

78. (canceled)

79: The exogenous genetic molecule of claim 69 comprising:
SEQ ID NO: 17;
a nucleic acid sequence with at least about 50% sequence identity to the nucleic acid sequence set forth in SEQ ID NO:17 or a functional fragment thereof; or
a nucleic acid sequence encoding a polypeptide with similar enzymatic activities and exhibiting at least about 50% sequence identity to a polypeptide encoded by the nucleic acid sequence set forth in SEQ ID NO:17 or a functional fragment thereof.

80. (canceled)

81: The exogenous genetic molecule of claim 69 comprising:
SEQ ID NO: 15;
a nucleic acid sequence with at least about 50% sequence identity to the nucleic acid sequence set forth in SEQ ID NO:15 or a functional fragment thereof; or
a nucleic acid sequence encoding a polypeptide with similar enzymatic activities and exhibiting at least about 50% sequence identity to a polypeptide encoded by the nucleic acid sequence set forth in SEQ ID NO:15 or a functional fragment thereof.

82-83. (canceled)

84: A process for the biosynthesis of 3-HP, derivatives thereof and/or compounds related thereto, said process comprising providing a means capable of producing 3-HP, derivatives thereof and/or compounds related thereto and producing 3-HP, derivatives thereof and/or compounds related thereto with said means.

85: A process for biosynthesis of 3-HP, and derivatives thereof, and compounds related thereto, said process comprising:
a step for performing a function of altering an organism capable of producing 3-HP, derivatives thereof, and/or compounds related thereto such that the altered organism produces more 3-HP, derivatives thereof, and/or compounds compared to a corresponding unaltered organism; and
a step for performing a function of producing 3-HP, derivatives thereof, and/or compounds related thereto in the altered organism.

86-87. (canceled)
